(19) United States
(12) Patent Application Publication
(10) Pub. No.: US 2023/0313121 A1
(43) Pub. Date: Oct. 5, 2023

(54) MODIFIED FILAMENTOUS FUNGI FOR PRODUCTION OF EXOGENOUS PROTEINS

(71) Applicant: DYADIC INTERNATIONAL (USA), INC., Jupiter, FL (US)

(72) Inventors: Marika Vitikainen, Espoo (FI); Anne Huuskonen, Espoo (FI); Andriy Kovalchuk, Espoo (FI); Christopher Landowski, Espoo (FI); Ronen Tchelet, Budapest (HU); Mark Aaron Emalfarb, Jupiter, FL (US); Markku Saloheimo, Espoo (FI)

(21) Appl. No.: 17/924,927

(22) PCT Filed: May 13, 2021
(86) PCT No.: PCT/IB2021/054082

§ 371 Date: Nov. 11, 2022

Related U.S. Application Data
(60) Provisional application No. 63/024,550, filed on May 14, 2020.

(51) Int. Cl. C12N 1/14; C12N 15/80; C12P 21/02

(52) U.S. Cl. CPC C12N 1/145; C12N 15/80; C12P 21/02; C12R 2001/645

(57) ABSTRACT

The present invention relates to genetically modified ascomycetous filamentous fungi, particularly of the species Thermothelomyces heterothallica, having reduced activity or expression of KEX2 and/or ALP7, said filamentous fungi is capable of producing elevated amounts and stability of an exogenous protein.

### FIELD OF THE INVENTION

The present invention relates to production of exogenous proteins in genetically modified ascomycetous filamentous fungi, in particular of the species Thermothelomyces heterothallica (formerly Myceliophthora thermophila), having reduced expression or activity of KEX2 and/or ALP7 proteases. The genetically modified ascomycetous filamentous fungi are used for robust production of highly stable proteins.

### BACKGROUND OF THE INVENTION

### Recombinant Protein Production

The expression and purification of recombinant proteins having functional post-translational protein modifications, such as glycosylation or phosphorylation, can only be achieved using eukaryotic expression systems. Eukaryotic protein expression systems, including mammalian cells, plant and fungi have become indispensable for the production of functional eukaryotic proteins.

Wild type Thermothelomyces heterothallica (Th. heterothallica) C1 (recently renamed from Myceliophthora thermophila, which in term was renamed from Chrysosporium lucknowense) is a thermotolerant ascomycetous filamentous fungus producing high levels of cellulases, which made it attractive for production of these and other proteins on a commercial scale.

For example, U.S. Pat. Nos. 8,268,585 and 8,871,493 to the Applicant of the present invention disclose a transformation system in the field of filamentous fungal hosts for expressing and secreting heterologous proteins or polypeptides. Also disclosed is a process for producing large amounts of polypeptides or proteins in an economical manner. The system comprises a transformed or transfected fungal strain of the genus Chrysosporium, more particularly of Chrysosporium lucknowense and mutants or derivatives thereof. Also disclosed are transformants containing Chrysosporium coding sequences, as well expressing-regulating sequences of Chrysosporium genes.

Wild type C1 was deposited in accordance with the Budapest Treaty with the number VKM F-3500 D, deposit date Aug. 29, 1996. High Cellulase (HC) and Low Cellulase (LC) strains have also been deposited, as described, for example, in U.S. Pat. No. 8,268,585.

Recently, the Applicant of the present application has shown that filamentous fungi, particularly Th. heterothallica is highly suitable for the production of secondary metabolites. International (PCT) Application No. PCT/IB2020/051015 discloses that Th. heterothallica is capable of producing cannabinoids and precursors thereof, particularly of producing cannabigerolic acid (CBGA) and/or cannabigerovarinic acid (CBGVA) and products thereof, including tetrahydrocannabinolic acid (THCA), cannabidiolic acid (CBDA) and cannabidivarinic acid (CBDVA), and use thereof for producing said precursors and cannabinoids.

International Application Publication No. WO/2015/004241 to Landowski et al. discloses multiple proteases deficient filamentous fungal cells and methods useful for the production of heterologous proteins.

### Coronavirus

Coronaviruses (CoVs) are the largest group of viruses belonging to the Nidovirales order, which includes Coronaviridae, Arteriviridae, and Roniviridae families. The Coronavirinae comprise one of two subfamilies in the Coronaviridae family, with the other being the Torovirinae. Coronaviruses are associated with illness from the common cold to more severe conditions such as Severe Acute Respiratory Syndrome (SARS-CoV) and Middle East Respiratory Syndrome (MERS-CoV). Severe acute respiratory syndrome coronavirus 2 (SARS-CoV-2) is the positive-sense, single-stranded RNA coronavirus that causes the coronavirus disease 2019 (COVID-19). Coronaviruses are zoonotic, meaning they are transmitted between animals and people. Common signs of coronavirus infection include respiratory symptoms, fever, coughing, shortness of breath and breathing difficulties. High concentrations of cytokines were recorded in plasma of critically ill patients infected with COVID-19. In more severe cases, infection can cause pneumonia, respiratory inflammation, severe acute respiratory syndrome, kidney failure and death. Recombinant production of viral proteins may be used as potential vaccine. Coronavirus spike proteins are considered as a major target for vaccine development.

There remains a need for expression systems for mass production of proteins that can be used in the pharmaceutical industry in an efficient and cost-effective way. In particular, there is a need for improved and robust expression systems that can produce stable antibodies as well as viral antigens for vaccination.

### SUMMARY OF THE INVENTION

The present invention provides genetically modified ascomycetous filamentous fungi having reduced expression of the proteases KEX2 and/or ALP7, capable of producing high amounts of highly stable proteins.

In particular, the present invention provides Thermothelomyces heterothallica strain C1 as an exemplary ascomycetous filamentous fungus genetically modified to enhance the production of exogenous proteins. In some embodiments, the fungi disclosed herein were modified to be deficient of fourteen proteases including KEX2 and ALP7.

Surprisingly, the present invention shows that Th. heterothallica, exemplifying ascomycetous filamentous fungi, can be genetically modified to significantly increase the expression and stability of exogenous proteins expressed by the Th. heterothallica cells compared to previous disclosed fungal strains. The present invention shows that the deletion of specific proteases including KEX2 or ALP7 significantly increase the stability of expressed proteins.

It is further disclosed that the combined deletion of KEX2 and ALP7 significantly increases the stability and amount of expressed exogenous proteins.

Advantageously, the genetically modified ascomycetous filamentous fungus of the invention was designed, in some embodiments, to produce secreted proteins, having reduced expression of secreted proteases. The secretion of the expressed proteins and the prevention of fragmented proteins in the medium simplify the purification procedure and increase the protein yield.

The exemplary Th. heterothallica C1 system of the present invention was engineered for production of protein of interest by disrupting genes encoding proteases naturally expressed by the fungus. Unexpectedly, the deletion of as many as thirteen or fourteen proteases did not disturb the fungi growth and proliferation rate, but at least maintain and even increased the growth rate, enabling a mass production of the exogenous protein.

Several Th. heterothallica C1 strains developed by the Applicant of the present invention are less sensitive to feedback repression by glucose and other fermentable sugars present in the growth medium as carbon source than conventional yeast strains and also most other ascomycetous filamentous fungal hosts, and consequently can tolerate higher feeding rate of the carbon source, leading to high yields production by this fungus.

In addition, some of the Th. heterothallica C1 strains developed by the Applicant of the present invention can be grown in liquid cultures with significantly reduced medium viscosity in fermenters, compared to most other ascomycetous filamentous fungal species. The low viscosity cultures of Th. heterothallica C1 are comparable to that of S. cerevisiae and other yeast species. The low viscosity may be attributed to the morphological change of the strain from having long and highly interlaced hyphae in the parental strain(s) to short and less interlaced hyphae in the developed strain(s). Low medium viscosity is highly advantageous in large scale industrial production.

According to one aspect, the present invention provides a filamentous fungus genetically modified to produce a protein of interest, the genetically modified filamentous fungus comprises at least one cell having reduced expression and/or protease activity of KEX2 and/or ALP7, the at least one cell comprising at least one exogenous polynucleotide encoding the protein of interest.

According to some embodiments, the ALP7 comprises an amino acid sequence having at least 75%, or at least 80%, or at least 85%, or at least 90%, or at least 95%, or at least 99%, or 100% identity to the amino acid sequence of Thermothelomyces heterothallica ALP7. According to certain embodiments, the Thermothelomyces heterothallica ALP7 comprises the amino acids of SEQ ID NO: 13.

According to some embodiments, the KEX2 comprises an amino acid sequence having at least 75%, or at least 80%, or at least 85%, or at least 90%, or at least 95%, or at least 99%, or 100% identity to the amino acid sequence of Thermothelomyces heterothallica KEX2. According to certain embodiments, the Thermothelomyces heterothallica KEX2 comprises the amino acids of SEQ ID NO: 14.

According to some embodiments, the modified filamentous fungus comprises at least one cell having reduced expression and/or activity of KEX2 and ALP7.

According to some embodiments, the modified filamentous fungus comprises at least one cell having reduced expression and/or activity of at least one additional protease.

According to some embodiments, the modified filamentous fungus comprises at least one cell having reduced expression and/or activity of at least 3, 4, 5, 6, 7, 8, 9, 10, 11, 12, 13, or 14 proteases. Each possibility represents a separate embodiment of the invention. According to certain embodiments, the modified filamentous fungus comprises at least one cell having reduced expression and/or activity of at least 5, 6, 7, 8, 9, 10, 11, 12, 13, or 14 proteases. Each possibility represents a separate embodiment of the invention

According to some embodiments, the at least one additional protease is selected from the group consisting of ALP1, PEP4, ALP2, PRT1, SRP1, ALP3, PEP1, MTP2, PEP5, MTP4, PEP6, and ALP4. Each possibility represents a separate embodiment of the invention.

According to some embodiments, the at least one additional protease is selected from the group consisting of ALP1, PEP4, ALP2, PRT1, SRP1, ALP3, PEP1, MTP2, PEP5, MTP4, PEP6, ALP4, ALP5, ALP6, SRP3, SRP5, and SRP8.

According to some embodiments, the at least one cell has reduced expression and/or activity of at least 2, 3, 4, 5, 6, 7, 8, 9, or 10 proteases selected from the group consisting of ALP1, PEP4, ALP2, PRT1, SRP1, ALP3, PEP1, MTP2, PEP5, MTP4, PEP6, ALP4, ALP5, ALP6, SRP3, SRP5, SRP8, and SRP10.

According to some embodiments, the modified filamentous fungus comprises at least one cell having reduced expression and/or activity of ALP1, PEP4, ALP2, PRT1, SRP1, ALP3, PEP1, MTP2, PEP5, MTP4, PEP6, ALP4 and ALP7. According to some embodiments, the modified filamentous comprises at least one cell having reduced expression and/or activity of ALP1, PEP4, ALP2, PRT1, SRP1, ALP3, PEP1, MTP2, PEP5, MTP4, PEP6, ALP4 and KEX2. According to some embodiments, the modified filamentous fungus further comprises at least one cell having reduced expression and/or activity of ALP1, PEP4, ALP2, PRT1, SRP1, ALP3, PEP1, MTP2, PEP5, MTP4, PEP6, ALP4, ALP7 and KEX2.

According to some embodiments, the filamentous fungus is further modified to produce proteins with N-glycans similar to those of human, companion animal and other mammalian proteins. According to some embodiments, the filamentous fungus comprises deletion or disruption of the alg3 gene such that the fungus fails to produce a functional alpha-1,3-mannosyltransferase. According to some additional or alternative embodiments, the filamentous fungus comprises deletion or disruption of the alg11 gene such that the fungus fails to produce a functional alpha-1,2-mannosyltransferase. According to yet further additional or alternative embodiments, the filamentous fungus is modified to over-express flippase. Flippase overexpression may be obtained by overexpression the fungus endogenous flippase or by expression of a heterologous flippase.

According to certain additional or alternative embodiments, the filamentous fungus further comprises expression of heterologous GlcNAc transferase 1 (GNT1) and GlcNAc transferase 2 (GNT2). In certain embodiments, the GNT1 comprises a heterologous Golgi localization signal.

According to some embodiments, the protein of interest is selected from the group consisting of an antigen, an antibody, an enzyme, a vaccine and a structural protein.

According to some embodiments, the protein of interest is a secreted protein. According to certain embodiments, the protein of interest has a leader or a signal peptide. According to other embodiments, the protein of interest is an intracellular protein.

According to some embodiments, the protein of interest includes two or more repetitive sequences of a protein or a protein fragment.

According to some embodiments, the protein of interest is fused to a tag. According to some embodiments, the tag is a C-terminal or N-terminal tag. According to some embodiments, the tag is selected from the group consisting of chitin binding protein (CBP), maltose binding protein (MBP), Strep-tag, glutathione-S-transferase (GST), FLAG-tag, Spytag, C-tag, ALFA-tag, V5-tag, Myc-tag, HA-tag, Spot-tag, T7-tag, NE-tag, and poly(His) tag. According to some embodiments, the tag is Spytag. According to some embodiments, the tag is C-tag.

According to some embodiments, the protein of interest is an antibody or a fragment thereof. According to certain embodiments, the antibody is IgG4 or IgG1. According to additional embodiments, the antibody is a bi- or multiple-specific antibody. According to specific embodiments, the antibody or fragment thereof is a neutralizing antibody against coronavirus.

According to some embodiments, the protein of interest is an anticalin.

According to some embodiment, the protein of interest is an FC-fusion protein.

According to some embodiments, the protein of interest is an antigen.

According to some embodiments, the protein of interest is a component of an infectious agent. According to some embodiments, the protein of interest is of a component of fungi, bacteria or viruses. According to some embodiments, the protein of interest is a viral component.

According to some embodiments, the viral component is of an epidemic virus. According to certain exemplary embodiments, the viral components is of a coronavirus, an influenza virus, hepatitis B, hepatitis C, papillomavirus, HIV, HTLV-1, or EBV.

According to some embodiments, the protein of interest is an influenza virus protein. According to certain embodiments, the protein of interest is hemagglutinin (HA) or a fragment thereof. According to certain embodiments, the protein of interest comprises the transmembrane domain (TMD) of hemagglutinin. According to specific embodiments, the protein of interest is of an influenza subtype H1N1.

According to some embodiments, the produced hemagglutinin protein is secreted.

According to some embodiments, the viral component is of a coronavirus. According to certain currently exemplary embodiments, the coronavirus is SARS-CoV-2 (COVID-19).

According to some embodiments, the protein of interest is a spike protein. According to some embodiments, the protein of interest comprises the receptor binding domain (RBD) sequence of SARS-CoV-2 spike protein or a fragment thereof. According to some embodiments, the protein of interest comprises the RBD of SARS-CoV-2 spike protein. According to some embodiments, the protein of interest consists of the RBD of SARS-CoV-2 spike protein. According to certain embodiments, the protein of interest comprises the receptor binding motif (RBM) of SARS-CoV-2 spike protein. According to specific embodiments, the RBD or fragment thereof is fused to a Spytag. According to certain embodiments, the protein of interest comprises two, three, or four repeats of RBD or a fragment thereof. According to additional embodiments, the protein of interest is nucleocapsid. According to some embodiments, the protein of interest is S2 fragment of the SARS-CoV-2 spike protein.

According to some embodiments, the protein of interest is a viral antigen fused to an Fc fragment. According to certain embodiments, the Fc is fused to the N terminus of the antigen. According to other embodiments, the Fc is fused to the C terminus of the antigen.

According to some embodiments, the protein of interest is Fc-RBD. According to other embodiments, the protein of interest is RBD-Fc.

According to some embodiments, the protein of interest comprises an amino acid sequence selected from the group consisting of SEQ ID NO: 45, SEQ ID NO: 47, SEQ ID NO: 49, SEQ ID NO: 51, SEQ ID NO: 53, SEQ ID NO: 55, and SEQ ID NO: 57.

According to some embodiments, the protein of interest is insulin. According to additional embodiments, the protein of interest is fibrinogen.

According to some embodiments, the protein of interest is a therapeutic protein.

According to some embodiments, the protein of interest is a vaccine protein antigen from rift valley fever virus (RVFV).

According to some embodiments, the protein of interest is a fusion protein comprised of two different antigens. According to some embodiments, the protein of interest is a fused protein comprised of two components of different viral antigens. According to certain embodiments, the viral antigens are of coronavirus and influenza virus.

According to some embodiments, the viral antigen is fused to an MHCII targeting sequence. According to certain embodiments, the viral antigen and the MHCII targeting sequence are connected via a linker.

In some embodiments, the tag is site-specific fluorescent labeling peptides/proteins.

According to some embodiments, the genetically modified ascomycetous filamentous fungus produces exogenous protein in an increased amount compared to the amount produced in a corresponding non-genetically modified parent ascomycetous filamentous fungus cultured under similar conditions. According to certain embodiments, the genetically modified ascomycetous filamentous fungus is capable of producing at least 2 times more exogenous protein compared to its parent strain.

According to some embodiments, the genetically modified ascomycetous filamentous fungus is capable of increasing the amount of a secreted exogenous protein in the growth medium by at least 1.5, 2, 5, or 10 times compared to its parent ascomycetous filamentous fungus. According to certain embodiments, the secreted protein is an intact protein.

According to some embodiments, the genetically modified ascomycetous filamentous fungus is capable of increasing the amount of an intracellular exogenous protein in the fungal cells by at least 1.5, 2, 5, or 10 times compared to its parent ascomycetous filamentous fungus.

According to some embodiments, the exogenous protein produced by the genetically modified ascomycetous filamentous fungus have an increased stability compared to a corresponding protein produced by the parent ascomycetous filamentous fungus strain cultured under similar conditions.

According to some embodiments, the genetically modified ascomycetous filamentous fungus grow at a higher rate compared to a corresponding parent ascomycetous filamentous fungus strain cultured under similar conditions.

The polynucleotide encoding the protein of interest may form part of a DNA construct or an expression vector.

According to some embodiments, the at least one exogenous polynucleotide is a DNA construct or an expression vector further comprising at least one regulatory element operable in said ascomycetous filamentous fungus. According to certain embodiments, the regulatory element is selected from the group consisting of a regulatory element endogenous to said fungus and a regulatory element heterologous to said fungus.

According to some embodiments, the ascomycetous filamentous fungus is of a genus within the group Pezizomycotina.

According to some embodiments, the ascomycetous filamentous fungus is of a genus selected from the group consisting of Thermothelomyces, Myceliophthora, Trichoderma, Aspergillus, Penicillium, Rasamsonia, Chrysosporium, Corynascus, Fusarium, Neurospora, and Talaromyces.

According to some embodiments, the ascomycetous filamentous fungus is of a species selected from the group consisting of Thermothelomyces heterothallica (also denoted Myceliophthora thermophila), Myceliophthora lutea, Aspergillus nidulans, Aspergillus funiculosus Aspergillus niger, Aspergillus oryzae, Trichoderma reesei, Trichoderma harzianum, Trichoderma longibrachiatum, Trichoderma viride, Rasamsonia emersonii. Penicillium chrysogenum, Penicillium verrucosum, Sporotrichum thermophile, Corynascus fumimontanus, Corynascus thermophilus, Chrysosporium lucknowense, Fusarium graminearum, Fusarium venenatum, Neurospora crassa, and Talaromyces piniphilus.

According to some embodiments, the ascomycetous filamentous fungus is a Thermothelomyces heterothallica strain comprising rDNA sequence having at least 95%, or at least 96%, or at least 97%, or at least 98%, or at least 99% or 100% identity to the nucleic acid sequence set forth in Sequence NO: 20.

According to some embodiments, the ascomycetous filamentous fungus is Thermothelomyces heterothallica C1.

According to an aspect, the present invention provides a method for producing a fungus capable of producing a protein of interest, the method comprising engineering the fungus to have inhibited or reduced expression and/or activity of KEX2 and/or ALP7.

According to some embodiments, the method comprises transforming at least one cell of the fungus with at least one exogenous polynucleotide.

According to an additional aspect, the present invention provides a method for producing a fungus capable of producing a protein of interest, the method comprising transforming at least one cell of the fungus with at least one exogenous polynucleotide, wherein said at least one cell having reduced expression and/or protease activity of KEX2 and/or ALP7.

According to some embodiments, the method comprises transforming the at least one cell of the fungus with at least two exogenous polynucleotides encoding for different proteins.

According to some embodiments, the method further comprises engineering the fungus to have inhibited or reduced expression and/or activity of at least one protease selected from the group consisting of ALP1, PEP4, ALP2, PRT1, SRP1, ALP3, PEP1, MTP2, PEP5, MTP4, PEP6, and ALP4 in the at least one cell.

According to certain embodiments, the method further comprises engineering the fungus to have inhibited or reduced expression and/or activity of at least two different proteases selected from the group consisting of ALP1, PEP4, ALP2, PRT1, SRP1, ALP3, PEP1, MTP2, PEP5, MTP4, PEP6, and ALP4.

According to some embodiments, inhibiting the expression of a protease comprising deleting or disrupting the endogenous gene encoding for the protease.

According to some embodiments, the ascomycetous filamentous fungus is of a genus within Pezizomycotina.

According to some embodiments, the ascomycetous filamentous fungus is of a genus selected from the group consisting of Thermothelomyces, Myceliophthora, Trichoderma, Aspergillus, Penicillium, Rasamsonia, Chrysosporium, Corynascus, Fusarium, Neurospora, and Talaromyces.

According to some embodiments, the ascomycetous filamentous fungus is of a species selected from the group consisting of Thermothelomyces heterothallica or (Myceliophthora thermophila), Myceliophthora lutea, Aspergillus nidulans, Aspergillus funiculosus, Aspergillus niger, Aspergillus oryzae, Trichoderma reesei, Trichoderma harzianum, Trichoderma longibrachiatum, Trichoderma viride, Rasamsonia emersonii, Penicillium chrysogenum, Penicillium verrucosum, Sporotrichum thermophile, Corynascus fumimontanus, Corynascus thermophilus, Chrysosporium lucknowense Fusarium graminearum, Fusarium venenatum, Neurospora crassa and Talaromyces piniphilus.

According to some embodiments, the ascomycetous filamentous fungus is a Thermothelomyces heterothallica strain comprising rDNA sequence having at least 95%, or at least 96%, or at least 97%, or at least 98%, or at least 99% or 100% identity to the nucleic acid sequence set forth in SEQ ID NO: 20.

According to some embodiments, the ascomycetous filamentous fungus is Thermothelomyces heterothallica C1.

According to a further aspect, the present invention provides a method of producing at least one protein of interest, the method comprising culturing the genetically modified fungus as described herein in a suitable medium; and recovering the at least one protein product.

According to some embodiments, the recovering step comprises recovering the protein from the growth medium, from the fungal mass or both.

According to some embodiments, the protein is recovered from the growth medium. According to certain embodiment, at least 50%, 60%, 70%, 80%, 90% or 95% of the protein is secreted.

According to some embodiments, the medium comprises a carbon source selected from the group consisting of glucose, sucrose, xylose, arabinose, galactose, fructose, lactose, cellobiose, glycerol and any combination thereof.

According to certain embodiments, culturing of the genetically modified fungus in a suitable medium provides for production of the protein of interest in an increased amount compared to the amount produced in a corresponding non-genetically modified parent fungus strain cultured under similar conditions.

According to certain embodiments, the corresponding parent fungus is of the same species of the genetically modified fungus. According to some embodiments, the corresponding parent fungus is isogenic to the genetically modified fungus.

According to another aspect, the present invention provides a protein of interest produced by any of the methods described herein.

According to an aspect, the present invention provides a protein of interest provided by a method comprising culturing the genetically modified fungus as described herein in a suitable medium; and recovering the protein of interest.

The protein of interest is as described hereinabove.

According to some embodiments, the protein of interest is a coronavirus antigen. According to some embodiments, the protein of interest is the coronavirus spike protein. According to certain embodiments, the protein of interest comprises a coronavirus RBD sequence or a fragment thereof. According to some embodiments, the protein of interest comprises the receptor binding motif (RBM) sequence of the coronavirus spike protein.

The present invention further provides a composition comprising two or more different protein of interest produced by any of the methods described herein.

According to certain embodiments, the composition comprises at least two different coronavirus antigens, said antigens comprises sequences of different coronavirus variants.

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution as are known in the art, with the stipulation that these variants and modifications must preserve the activity of protein or enzymes described herein.

It is to be understood that any combination of each of the aspects and the embodiments disclosed herein is explicitly encompassed within the disclosure of the present invention.

Other objects, features and advantages of the present invention will become clear from the following description and drawings.

### BRIEF DESCRIPTION OF THE FIGURES

FIG. 1. Shows Western blotting with C-tag detection from 24-well plate cultures of C1 transformants producing either RBD-C-tag (left panel) or RBD-Spytag-C-tag (right panel).

FIG. 2. Production of RBD-C-tag and RBD-Spytag-C-tag in C1 protease deficient strains from which 12-14 protease genes are deleted. Highest production of RBD proteins is in DNL155 and DNL159 strains from which kex2 is deleted. One of the three parallel clones of both RBD-C-tag and RBD-Spytag-C-tag were growing poorly and thus produced lower protein levels.

FIGS. 3A-3B. C-tag affinity purification of RBD-C-tag from a bioreactor cultivation of C1 strain M4169. Stained SDS gel (FIG. 3A) and Western (FIG. 3B) analysis of samples from different purification steps are shown. Start=Start sample after clarification, diluted 1:5 in gel; Flow start=flowthrough in the beginning of sample loading, diluted 1:5 in gel; Flow end=flowthrough in the end of sample loading, diluted 1:5 in gel; Fr4-F9=elution fractions. Note that migration of the elution samples before dialysis is not normal because of high MgCl₂ concentration.

FIG. 4. Schematic description of the C1 lineage.

FIG. 5. shows spiking experiments with antibodies in different protease deficient strains. C1 protease deletion strains were cultivated in 24-cell culture plates for 4 days. For spiking experiments, an antibody was incubated in the culture supernatants, Samples were taken from the samples at different times (0 h, 3 h, o/n, and o/2n) and analyzed by western blot. Separate antibodies were used for detection of heavy and light chains. 270 ng of mAb were loaded in each lane. Control—200 ng.

FIG. 6. shows spiking experiments with antibodies in different 13×protease deficient strains. C1 protease deletion strains were cultivated in 24-cell culture plates for 4 days. For spiking experiments, an antibody was incubated in the culture supernatants, Samples were taken from the samples at different times (0 h, 3 h, o/n, and o/2n) and analyzed by western blot. 270 ng of mAb were loaded in each lane. Control—200 ng.

FIG. 7. shows spiking experiments with fibrinogen in different 13×protease deficient strains. C1 protease deletion strains were cultivated in 24-cell culture plates for 4 days. For spiking experiments, fibrinogen protein was incubated in the culture supernatants, Samples were taken from the samples at different times (0 h, 3 h, o/n, and o/2n) and analyzed by western blot. Polyclonal anti-fibrinogen antibodies (all fibrinogen chains) were used in detection. 240 ng of fibrinogen were loaded in each lane. Control—200 ng.

FIG. 8. shows spiking experiments with Fc-FGF21 in different 13× protease deficient strains. C1 protease deletion strains were cultivated in 24-cell culture plates for 4 days. For spiking experiments, Fc-FGF21 was incubated in the culture supernatants, Samples were taken from the samples at different times (0 h, 4 h, o/n, and o/2n) and analyzed by western blot. Two antibodies (anti-Fc and Anti-FGF21) were used in detection. 240 ng of Fc-FGF21 were loaded in each lane. Control—200 ng.

FIG. 9. shows spiking (left panel) and expression (right panel) of mAb in 12× proteases vs. 13× proteases deficient strains as indicated.

FIG. 10. shows mAb expression in 12× and 13× proteases deficient strains. Expression construct of a mAb was transformed into the 13× protease deletion strain with kex2 deletion. Transformants were grown in 24-well plates and produced mAbs were analyzed by Western blot. The same mAb expressed in the parental 12× protease deletion strain and in the 13×Δalp7 deletion strain are shown as controls.

FIG. 11. shows production of an antigen protein of rvfv under bgl promoter by 14×protease deficient strain dnl155 and 13× proteases deficient strain as indicated.

FIGS. 12A-12B show that RBD-Spytag and conjugation of RBD-SpyTag with SpyCatcher HBsAg VLP generate trimers and/or dimers. FIG. 12A—Western blot. FIG. 12B—SDS-PAGE.

FIGS. 13A-13F. show binding of soluble and conjugated RBD to hACE-2 by indirect ELISA. FIG. 13A—a schematic representation of the binding of anti-RBD CR3022 antibody to RBD-ST:SC-HBsAg VLP particle and the detection by labeled goat α-human IgG-AP. FIG. 13B—Detection of different batches of RBD with or without the VLP particle. FIGS. 13C-13D—schematic representation of the binding of RBD-ST:SC-HBsAg VLP to hACE (13C) and control (13D). FIG. 13E-13F. ELISA results of binding hACE to VLP-RBD in conjugated protein (13E) or only VLP (13F).

FIGS. 14A-14B. Western analysis of C1 transformants producing the RBD-Fc (FIG. 14A) or Fc-RBD (FIG. 14B) fusion protein. The parental strains used for production are shown. DNL155 strain is shown as a negative control. The lanes numbered 1-12 correspond to individual transformants.

FIG. 15.—Western blotting with C-tag detection from 24-well plate cultures of C1 transformants producing recombinant antigen αMHCII-Cal07 under control of either endogenous C1 bgl8 promoter or synthetic AnSES promoter in transformants derived from DNL155 and M3599 strains. The gel mobility of the target protein agrees with its predicted size of 87 kDa. Additionally, an endogenous C1 background protein of the size of 70 kDa reacting with the antibody present in all DNL155-derived parental strain derived samples.

FIGS. 16A-16C—C-tag affinity purification of αMHCII-Cal07 from a bioreactor cultivation of C1 strain M4540. Stained SDS gel (FIG. 16A) and Western (FIG. 16B) analysis of samples from different purification steps are shown. Input=Start sample after clarification, diluted 1:10 in gel; Flowthrough=flowthrough in the beginning of sample loading, diluted 1:10 in gel; Wash=flowthrough during column wash. Note that migration of the elution samples before dialysis is not normal because of high MgCl2 concentration. FIG. 16C—stained SDS-PAGE gel and Western blot analysis of αMHCII-Cal07 sample after dialysis in comparison with the reference protein.

FIG. 17—Western blotting result from 24-well plate culture of C1 transformants producing RBD variants. Yellow colour is the overlay signal of both anti-RBD (red signal) and anti-C-tag (green signal) detection agents. UK is RBD_B.1.1.7-UK, SA is RBD_B.1.351-SA and BR is RBD_1.1.28.1(P.1)-BR. The sample denoted Wuhan is from the M4169 C1 strain producing Wuhan RBD (Example 4).

### DETAILED DESCRIPTION OF THE INVENTION

The present invention provides alternative, highly efficient system for producing high amounts of proteins. The system of the invention is based in part on the filamentous fungus Thermothelomyces heterothallica C1 and particular strains thereof, which have been previously developed as a natural biological factory for protein as well as secondary metabolite production. These strains show high growth rate while keeping low culture viscosity, and are thus highly suitable for continuous growth in fermentation cultures at volumes as high as 100,000-150,000 liters or greater. The present invention in some embodiments provides genetically modified fungi having reduced expression and/or activity of KEX2 and/or ALP7.

### Definitions

Ascomycetous filamentous fungi as defined herein refer to any fungal strain belonging to the group Pezizomycotina. The Pezizomycotina comprises, but is not limited to the following groups:

Sordariales, including genera:
Thermothelomyces (including species: heterothallica and thermophola),
Myceliophthora (including the species lutea and unnamed species),
Corynascus (including the species fumimontanus),
Neurospora (including the species crassa);

Hypocreales, including genera:
Fusarium (including the species graminearum and venenatum),
Trichoderma (including the species reesei, harzianum, longibrachiatum and viride);

Onygenales, including genera:
Chrysosporium (including the species lucknowense);

Eurotiales, including genera:
Rasamsonia (including the species emersonii),
Penicillium (including the species verrucosum),
Aspergillus (including the species funiculosus, nidulans, niger and oryzae)
Talaromyces (including the species piniphilus (formerly Penicillium funiculosum));

It is to be understood that the above list is not conclusive, and is meant to provide an incomplete list of industrially relevant filamentous ascomycetous fungal species.

While there may be filamentous ascomycetous species outside Pezizomycotina, that group does not contain Saccharomycotina, which contains most commonly known non-filamentous industrially relevant genera, such as Saccharomyces, Komagataella (including formerly Pichia pastoris), Kluyveromyces or Taphrinomycotina, which contains some other commonly known non-filamentous industrially relevant genera, such as Schizosaccharomyces.

All taxonomical categories above are defined according to the NCBI Taxonomy browser (ncbi.nlm.nih.gov/taxonomy) as of the date of the patent application.

It must be appreciated that fungal taxonomy is in constant move, and the naming and the hierarchical position of taxa may change in the future. However, a skilled person in the art will be able to unambiguously determine if a particular fungal strain belongs to the group as defined above.

According to certain embodiments, the filamentous fungus genus is selected from the group consisting of Myceliophthora, Thermothelomyces, Aspergillus, Penicillium, Trichoderma, Rasamsonia, Chrysosporium, Corynascus, Fusarium, Neurospora, Talaromyces and the like. According to some embodiments, the fungus is selected from the group consisting of Myceliophthora thermophila, Thermothelomyces thermophila (formerly M. thermophila), Thermothelomyces heterothallica (formerly M. thermophila and heterothallica), Myceliophthora lutea, Aspergillus nidulans, Aspergillus funiculosus Aspergillus niger, Aspergillus oryzae, Penicillium chrysogenum, Penicillium verrucosum, Trichoderma reesei, Trichoderma harzianum, Trichoderma longibrachiatum, Trichoderma viride, Chrysosporium lucknowense, Rasamsonia emersonii, Sporotrichum thermophile, Corynascus fumimontanus, Corynascus thermophilus, Fusarium graminearum, Fusarium venenatum, Neurospora crassa, and Talaromyces piniphilus.

In particular, the present invention provides Thermothelomyces heterothallica strain C1 as model for an ascomycetous filamentous fungus, capable of producing high amounts of stable proteins.

The terms “Thermothelomyces” and its species “Thermothelomyces heterothallica and thermophila” are used herein in the broadest scope as is known in the art. Description of the genus and its species can be found, for example, in Marin-Felix Y (2015. Mycologica 107(3): 619-632 doi.org/10.3852/14-228) and van den Brink J et al. (2012, Fungal Diversity 52(1):197-207). As used herein “C1” or “Thermothelomyces heterothallica C1” or Th. heterothallica C1, or C1 all refer to Thermothelomyces heterothallica strain C1.

It is noted that the above authors (Main-Felix et al., 2015) proposed splitting of the genus Myceliophthora based on differences in optimal growth temperature, morphology of the conidiospore, and details of the sexual reproduction cycle. According to the proposed criteria C1 clearly belongs to the newly established genus Thermothelomyces, which contain former thermotolerant Myceliophthora species rather than to the genus Myceliophthora, which remains to include the non-thermotolerant species. As C1 can form ascospores with some other Thermothelomyces (formerly Myceliophthora) strains with opposite mating type, C1 is best classified as Th. heterothallica strain C1, rather than Th. thermophila C1.

It must also be appreciated that the fungal taxonomy was also in constant change in the past, so the current names listed above may be preceded by a variety of older names beyond Myceliophthora thermophila (van Oorschot, 1977. Persoonia 9(3):403), which are now considered synonyms. For example, Thermothelomyces heterothallica (Marin-Felix et al., 2015. Mycologica, 3:619-63), is synonymized with Corynascus heterotchallicus, Thielavia heterothallica, Chrysosporium lucknowense and thermophile as well as Sporotrichium thermophile (Alpinis 1963. Nova Hedwigia 5:74).

It is further to be explicitly understood that the present invention encompasses any strain containing a ribosomal DNA (rDNA) sequence that shows 99% homology or more to SEQ ID NO: 20, and all those strains are considered to be conspecific with Thermothelomyces heterothallica.

Particularly, the term Th. heterothallica strain C1 encompasses genetically modified sub-strains derived from the wild type strain, which have been mutated, using random or directed approaches, for example, using UV mutagenesis, or by deleting one or more endogenous genes. For example, the C1 strain may refer to a wild type strain modified to delete one or more genes encoding an endogenous protease. For example, C1 strains which are encompassed by the present invention include strain UV18-25, deposit No. VKM F-3631 D; strain NG7C-19, deposit No. VKM F-3633 D; and strain UV13-6, deposit No. VKM F-3632 D. Further C1 strain that may be used according to the teachings of the present invention include HC strain UV18-100f, deposit No. CBS141147; HC strain UV18-100f, deposit No. CBS141143; LC strain W1L #100I, deposit No. CBS141153; and LC strain W1L #100I, deposit No. CBS141149 and derivatives thereof.

It is to be explicitly understood that the teachings of the present invention encompass mutants, derivatives, progeny, and clones of the Th. heterothallica C1 strains, as long as these derivatives, progeny, and clones, when genetically modified according to the teachings of the present invention are capable of producing at least one protein product according to the teachings of the invention.

It is to be explicitly understood that the term “derivative” with reference to fungal line encompasses any fungal parent line with modifications positively affecting product yield, efficiency, or efficacy, or affecting any trait improving the fungal derivative as a tool to produce the desired protein. As used herein, the term “progeny” refers to an unmodified or partially modified descendant from the parent fungal line, such as cell from cell. The term “parent strain” refers to a corresponding fungal strain not having reduced expression or activity of specific protease according to the invention.

According to an aspect of the present invention there is provided a genetically modified filamentous fungus for producing a protein of interest, the genetically modified filamentous fungus comprises at least one cell having reduced or abolished expression and/or activity of the protease KEX2 and/or ALP7 and at least one additional protease, said filamentous fungus comprises at least one cell comprising at least one exogenous polynucleotide encoding the protein of interest.

According to an aspect of the present invention there is provided a genetically modified filamentous fungus for producing heterologous protein, the genetically modified filamentous fungus comprises at least one cell having reduced or abolished expression and/or activity of KEX2 and at least one additional protease, said filamentous fungus comprises at least one cell comprising at least one exogenous polynucleotide encoding a heterologous protein.

According to an aspect of the present invention there is provided a genetically modified filamentous fungus for producing heterologous protein, the genetically modified filamentous fungus comprises at least one cell having reduced or abolished expression and/or activity of ALP7 and at least one additional protease, said filamentous fungus comprises at least one cell comprising at least one exogenous polynucleotide encoding a heterologous protein.

According to an aspect of the present invention there is provided a genetically modified filamentous fungus for producing heterologous protein, the genetically modified filamentous fungus comprises at least one cell having reduced or abolished expression and/or activity of the proteases ALP7, KEX2 and at least one additional protease, said filamentous fungus comprises at least one cell comprising at least one exogenous polynucleotide encoding a heterologous protein.

According to some embodiments, the at least one cell having reduced or abolished expression and/or activity of 13 proteases, in which one of them is KEX2. According to some embodiments, the at least one cell having reduced or abolished expression and/or activity of 13 proteases, in which one of them is ALP7. According to some embodiments, the at least one cell having reduced or abolished expression and/or activity of 14 proteases, including KEX2 and ALP7.

The terms “protein” and “polypeptide” are used herein interchangeably and refer to a polymer of amino acids and do not refer to a specific length of the product; thus, peptides, oligopeptides, and polypeptide are included within this definition.

As used herein, the term “protein of interest” refers to a protein that is desirably expressed in filamentous fungi at high levels. Such proteins include but not limited to antibodies, enzymes, substrate binding proteins, structural proteins, antigens and the like.

According to some embodiments, the ascomycetous filamentous fungus comprises at least one cell having reduced or abolished expression and/or activity of KEX2 and at least one more protease.

According to certain embodiments, the ascomycetous filamentous fungus comprises at least one cell having reduced or abolished expression and/or activity at least three, at least four, at least five, at least six, at least seven, at least eight, at least nine, at least ten, at least eleven, at least twelve, at least thirteen, at least fourteen or at least fifteen proteases.

According to some embodiments, the genetically modified filamentous fungus does not express KEX2. According to some embodiments, the genetically modified filamentous fungus does not express ALP7.

According to some embodiments, the genetically modified filamentous fungus does not express ALP1. According to some embodiments, the genetically modified filamentous fungus does not express PEP4. According to some embodiments, the genetically modified filamentous fungus does not express ALP2. According to some embodiments, the genetically modified filamentous fungus does not express PRT1. According to some embodiments, the genetically modified filamentous fungus does not express SRP1. According to some embodiments, the genetically modified filamentous fungus does not express ALP3. According to some embodiments, the genetically modified filamentous fungus does not express PEP1. According to some embodiments, the genetically modified filamentous fungus does not express MTP2. According to some embodiments, the genetically modified filamentous fungus does not express PEP5. According to some embodiments, the genetically modified filamentous fungus does not express MTP4. According to some embodiments, the genetically modified filamentous fungus does not express PEP6. According to some embodiments, the genetically modified filamentous fungus does not express ALP4.

According to specific embodiments, the ascomycetous filamentous fungus comprises at least one cell having reduced or abolished expression and/or activity of at least one additional protease selected from the group consisting of ALP1, PEP4, ALP2, PRT1, SRP1, ALP3, PEP1, MTP2, PEP5, MTP4, PEP6, and ALP4. Each possibility represents a separate embodiment of the invention.

According to an aspect of the present invention there is provided a genetically modified ascomycetous filamentous fungus for producing a protein of interest, wherein the genetically modified filamentous fungus comprises at least one cell comprising exogenous polynucleotides encoding for the protein of interest, said genetically modified ascomycetous filamentous fungus does not express or expresses reduced amount of KEX2 and/or ALP7, and at least one additional protease selected form the group consisting of ALP1, PEP4, ALP2, PRT1, SRP1, APL3, PEP1, MTP2, PEP5, MTP4, PEP6, and ALP4.

According to some embodiments, the filamentous fungus does not express or expresses reduced amount of KEX2, ALP1, PEP4, ALP2, PRT1, SRP1, ALP3, PEP1, MTP2, PEP5, MTP4, PEP6, and ALP4.

According to some embodiments, the filamentous fungus does not express or expresses reduced amount of ALP7, ALP1, PEP4, ALP2, PRT1, SRP1, ALP3, PEP1, MTP2, PEP5, MTP4, PEP6, and ALP4.

According to some embodiments, the filamentous fungus does not express or expresses reduced amount of KEX2, ALP7, ALP1, PEP4, ALP2, PRT1, SRP1, ALP3, PEP1, MTP2, PEP5, MTP4, PEP6, and ALP4.

According to an aspect, the present invention provides a genetically modified ascomycetous filamentous fungus for producing a viral antigen, wherein the genetically modified filamentous fungus comprises at least one cell comprising exogenous polynucleotides encoding for the viral antigen, said genetically modified ascomycetous filamentous fungus does not express or expresses reduced amount of KEX2, ALP7, ALP1, PEP4, ALP2, PRT1, SRP1, ALP3, PEP1, MTP2, PEP5, MTP4, PEP6, and ALP4.

According to some embodiments, the viral antigen is a vaccine antigen protein from rift valley fever virus (RVFV).

According to an aspect, the present invention provides a genetically modified ascomycetous filamentous fungus for producing a receptor binding domain (RBD) of SARS-CoV2 spike domain, wherein the genetically modified filamentous fungus comprises at least one cell comprising exogenous polynucleotides encoding for the RBD, said genetically modified ascomycetous filamentous fungus does not express or expresses reduced amount of KEX2, ALP7, ALP1, PEP4, ALP2, PRT1, SRP1, ALP5, PEP1, MTB2, PEP5, MTP4, PEP6, and ALP4.

The kex2 gene, also known as qds1, srb1, and vmn45, encodes for KEX2 or KEXIN protease. The KEX2 protease is a serine peptidase. The Thermothelomyces heterothallica KEX2 amino acid sequence is set forth in SEQ ID NO: 14.

According to some embodiments, the KEX2 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 14.

The Thermothelomyces heterothallica ALP7 amino acid sequence is set forth in SEQ ID NO: 13.

According to some embodiments, the ALP7 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 13.

The alp1 gene encode for alkaline protease 1. ALP1 is a secreted alkaline protease that allows assimilation of proteinaceous substrates. The Thermothelomyces heterothallica ALP1 amino acid sequence is set forth in SEQ ID NO: 1.

According to some embodiments, the ALP1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 1.

The pep4 gene (aliases: pho9, pra1, yscA) is an aspartic peptidase. The Thermothelomyces heterothallica PEP4 amino acid sequence is set forth in SEQ ID NO: 2.

According to some embodiments, the PEP4 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 2.

The Thermothelomyces heterothallica ALP2 amino acid sequence is set forth in SEQ ID NO: 3.

According to some embodiments, the ALP2 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 3.

The Thermothelomyces heterothallica PRT1 amino acid sequence is set forth in SEQ ID NO: 4.

According to some embodiments, the PRT1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 4.

The Thermothelomyces heterothallica SRP1 amino acid sequence is set forth in SEQ ID NO: 5.

According to some embodiments, the SRP1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 5.

The Thermothelomyces heterothallica ALP3 amino acid sequence is set forth in SEQ ID NO: 6.

According to some embodiments, the ALP3 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 6.

The Thermothelomyces heterothallica PEP1 amino acid sequence is set forth in SEQ ID NO: 7.

According to some embodiments, the PEP1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 7.

The Thermothelomyces heterothallica MTP2 amino acid sequence is set forth in SEQ ID NO: 8.

According to some embodiments, the MTP2 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 8.

The Thermothelomyces heterothallica PEP5 amino acid sequence is set forth in SEQ ID NO: 9.

According to some embodiments, the PEP5 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 9.

The Thermothelomyces heterothallica MTP4 amino acid sequence is set forth in SEQ ID NO: 10.

According to some embodiments, the MTP4 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 10.

The Thermothelomyces heterothallica PEP6 amino acid sequence is set forth in SEQ ID NO: 11.

According to some embodiments, the PEP6 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 11.

The Thermothelomyces heterothallica ALP4 amino acid sequence is set forth in SEQ ID NO: 12.

According to some embodiments, the ALP4 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 12.

The Thermothelomyces heterothallica ALP5 amino acid sequence is set forth in SEQ ID NO: 15.

According to some embodiments, the ALP5 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 15.

The Thermothelomyces heterothallica ALP6 amino acid sequence is set forth in SEQ ID NO: 16.

According to some embodiments, the ALP6 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 16.

The Thermothelomyces heterothallica SRP3 amino acid sequence is set forth in SEQ ID NO: 17.

According to some embodiments, the SRP3 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 17.

The Thermothelomyces heterothallica SRP5 amino acid sequence is set forth in SEQ ID NO: 18.

According to some embodiments, the SRP5 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 18.

The Thermothelomyces heterothallica SRP8 amino acid sequence is set forth in SEQ ID NO: 19.

According to some embodiments, the SRP8 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 19.

According to some embodiments, the protein of interest is fused to a tag. According to some embodiments, the tag is a C-terminal or N-terminal tag. According to some embodiments, the tag is selected from the group consisting of chitin binding protein (CBP), maltose binding protein (MBP), Strep-tag, glutathione-S-transferase (GST), FLAG-tag, Spytag, C-tag, ALFA-tag, V5-tag, Myc-tag, HA-tag, Spot-tag, T7-tag, NE-tag, and poly(His) tag. According to some embodiments, the tag is Spytag. According to some embodiments, the tag is C-tag.

As used herein, the term “tag” refers to an amino acid sequence, which is typically in the art fused to or included in another amino acid sequence for a) facilitating purification of the overall amino acid sequence or polypeptide, b) improving expression of the overall amino acid sequence or polypeptide, and/or c) facilitating detection of the overall amino acid sequence or polypeptide.

The term “C-tag” is well known in the art and refers to a 4 amino acid affinity tag: E-P-E-A (glutamic acid-proline-glutamic acid-alanine), which can be fused at the C-terminus of any recombinant protein. The tag offers high affinity and selectivity when used for purification purposes.

The term “Spytag” is well known in the art and refers to a short peptide which binds covalently to SpyCatcher protein. Spytag sequence is Ala-His-Ile-Val-Met-Val-Asp-Ala-Tyr-Ly s-Pro-Thr-Lys.

The term “Strep-tag” is used herein as known in the art and refers to a method which allows the purification and detection of proteins by affinity chromatography. The method is based on the Strep-Tactin connection.

The term “Glutathione S-transferases (GSTs)” is used herein as known in the art and is based on the strong binding affinity of the GST protein to glutathione (GSH). A GST-tag is often used to separate and purify proteins that contain the GST-fusion protein. The tag is 220 amino acids in length.

The term “FLAG-tag” is used herein as known in the art and refers to a polypeptide protein tag that can be added to a protein using recombinant DNA technology. It is one of the most specific tags and it is an artificial antigen to which specific, high affinity monoclonal antibodies have been developed and hence can be used for protein purification by affinity chromatography.

The term “ALFA-tag” is used herein as know in the art and refers to an epitope tag that is specifically recognized by a nanobody that can be used for detection and purification.

The V5-tag is a short peptide tag for detection and purification of proteins. The V5 tag can be fused/cloned to a recombinant protein and detected in ELISA, flow cytometry, immunoprecipitation, immunofluorescence, and Western blotting with antibodies and Nanobodies.

The term “Myc-tag” is used herein as known in the art and refers to a short peptide tag derived from the c-myc gene that can be recognized by specific antibodies.

The “HA-tag” is used herein as known in the art and refers to a peptide derived from the Human influenza hemagglutinin (HA) molecule, corresponding to amino acids 98-106. This tag is use to facilitate the detection, isolation, and purification of a protein of interest.

The “Spot-tag” is a 12-amino acid peptide tag recognized by a single-domain antibody nanobody (sdAb). The tag can be used to a variety of applications including: immunoprecipitation, affinity purification, immunofluorescence, and super-resolution microscopy.

The term “T7 tag” is used herein as known in the art and refers to an epitope tag composed of an 11-residue peptide encoded from the leader sequence of the T7 bacteriophage gene 10.

The term “NE-tag” is used herein as known in the art and refers to a synthetic peptide tag (NE tag) designed as an epitope tag for detection, quantification and purification of recombinant proteins. This peptide tag is composed of eighteen hydrophilic amino acids.

The term “poly(His) tag” or “polyhistidine-tag” is as known in the art and refers to an amino acid motif in proteins that typically consists of at least six histidine (His) residues, often at the N- or C-terminus of the protein. It is also known as hexa histidine-tag, 6×His-tag, and His6 tag. The short peptide can be bound by metal ions such as divalent nickel or cobalt.

According to some embodiments, the filamentous fungus is further modified to produce proteins with N-glycans similar to those of human, companion animal and other mammalian proteins. According to some embodiments, the filamentous fungus comprises deletion or disruption of the alg3 gene such that the fungus fails to produce a functional alpha-1,3-mannosyltransferase. According to some embodiments, filamentous fungus comprises deletion or disruption of the alg11 gene such that the fungus fails to produce a functional alpha-1,2-mannosyltransferase. According to some embodiments, the filamentous fungus comprises over-expression of an endogenous flippase or expression of a heterologous flippase.

According to certain embodiments, the filamentous fungus further comprises expression of heterologous GlcNAc transferase 1 (GNT1) and GlcNAc transferase 2 (GNT2). In certain embodiments, the GNT1 comprises a heterologous Golgi localization signal. In some embodiments, the heterologous GNT1 and GNT2 are animal-derived.

According to some embodiments, the protein of interest is an antigen. According to some embodiments, the protein of interest is a spike protein. According to some embodiments, the protein of interest comprises the receptor binding domain (RBD) sequence of SARS-CoV-2 spike protein or a fragment thereof. According to some embodiments, the protein of interest is the RBD of SARS-CoV-2 spike protein. According to certain embodiments, the protein of interest comprises the receptor binding motif (RBM) of SARS-CoV-2 spike protein. According to some embodiments, the protein of interest comprises the glycoprotein-binding domain (GBD) sequence of the SARS-CoV-2 S protein. According to specific embodiments, the RBD or fragment thereof is fused to a Spytag. According to certain embodiments, the RBD or fragment thereof is fused to C tag. According to additional embodiments, the RBD is fused to an Fc of an antibody. According to certain embodiments, the protein of interest comprises two, three, or four repeats of RBD or a fragment thereof.

The coronavirus antigen sequence can be manipulated according to any known or discovered variant of the coronavirus. For example, the sequence can be manipulated according to a sequence described in Rambaut et al. nCoV-2019 Genomic Epidemiology, December 2020 (https://virological.org/t/preliminary-genomic-characterisation-of-an-emergent-sars-cov-2-lineage-in-the-uk-defined-by-a-novel-set-of-spike-mutations/563), Tegally, H. et al. 2020 (https://www.medrxiv.org/content/10.1101/2020. 12.21.2024 8640v1), or Faria N R, et al. 2020 (https://virological.org/t/genomic-characterisation-of-an-emergentsars-cov-2-lineage-in-manaus-preliminary-findings/586). The present invention encompasses amino acid sequences that are substantially homologous to amino acids sequences based on any one of the sequences identified in this application. The terms “sequence identity” and “sequence homology” are considered synonymous in this specification.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g. GAP, BESTFIT, FASTA or TFASTA), or BLAST and BLAST 2.0 algorithms.

In a comparison, the identity may exist over a region of the sequences that is at least 10 amino acid residues in length (e.g. at least 15, 20, 30, 40, 50, 75, 100, 150, 200, 250, 300, 350, 400, 450, 500, 550, 600, 650 or 685 amino acid residues in length, e.g. up to the entire length of the reference sequence). Each possibility represents a separate embodiment of the invention.

The term “exogenous” as used herein refers to a polynucleotide or protein which is not naturally expressed within the fungus (e.g., heterologous polynucleotide from a different species). The exogenous polynucleotide may be introduced into the fungus in a stable or transient manner, so as to produce a ribonucleic acid (RNA) molecule and/or a polypeptide molecule.

The term “heterologous” as used herein includes a sequence that was inserted to the fungi and is not naturally found in the fungi.

The terms “DNA construct”, “expression vector”, “expression construct” and “expression cassette” are used to refer to an artificially assembled or isolated nucleic acid molecule which includes a nucleic acid sequence encoding a protein of interest and which is assembled such that the protein of interest is functionally expressed in a target host cell. An expression vector typically comprises appropriate regulatory sequences operably linked to the nucleic acid sequence encoding the protein of interest. An expression vector may further include a nucleic acid sequence encoding a selection marker.

The terms “polynucleotide”, “nucleic acid sequence”, and “nucleotide sequence” are used herein to refer to polymers of deoxyribonucleotides (DNA), ribonucleotides (RNA), and modified forms thereof in the form of a separate fragment or as a component of a larger construct. A nucleic acid sequence may be a coding sequence, i.e., a sequence that encodes for an end product in the cell, such as a protein.

A sequence (such as, nucleic acid sequence and amino acid sequence) that is “homologous” to a reference sequence refers herein to percent identity between the sequences, where the percent identity is at least 70%, at least 75%, preferably at least 80%, at least 85%, at least 90%, at least 95%, at least 98% at least 99% or at least 99.5%. Each possibility represents a separate embodiment of the present invention. Homologous nucleic acid sequences include variations related to codon usage and degeneration of the genetic code.

Nucleic acid sequences encoding the proteins of the present invention may be optimized for expression. Examples of such sequence modifications include, but are not limited to, an altered G/C content to more closely approach that typically found in filamentous fungi.

The phrase “codon optimization” refers to the selection of appropriate DNA nucleotides for use within a structural gene or fragment thereof that approaches codon usage within the organism of interest, and/or to a process of modifying a nucleic acid sequence for enhanced expression in the host cells of interest by replacing at least one codon (e.g., one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Therefore, an optimized gene or nucleic acid sequence refers to a gene in which the nucleotide sequence of a native or naturally occurring gene has been modified in order to utilize statistically-preferred or statistically-favored codons within the organism.

Sequence identity may be determined using a nucleotide/amino acid sequence comparison algorithm, as known in the art.

The term “coding sequence” is used herein to refer to a sequence of nucleotide starting with a start codon (ATG) containing any number of codons excluding stop codons, and a stop codon (TAA, TGA, TAA), which code for a functional polypeptide.

Any coding sequence, or amino acid sequence listed herein also encompasses truncated sequences, which are missing 1, 2, 3, 4, 5, 10, 15, 20, 25, 50, or more codons or amino acids from any part of the sequence. Truncated versions of coding sequences or amino sequences can be identified using nucleotide/amino acid sequence comparison algorithm, as known in the art.

Any coding sequence, or amino acid sequence listed herein also encompasses fused sequences, which contain besides the coding sequence provided herein, or a truncation of that sequence as defined above, other sequences. The fused sequences can be sequences as disclosed herein and other sequences. Fused coding sequences or amino sequences can be identified using nucleotide/amino acid sequence comparison algorithm, as known in the art.

DNA sequences are assembled to expression cassettes, selection cassettes and further to DNA constructs and/or expression vectors by conventional molecular biological approaches utilizing restriction endonucleases and ligases, Gibson assembly or yeast recombination. Also, the above can be synthesized by DNA synthesis service providers. As known in the art, several different techniques can achieve the same result.

DNA sequences are assembled to expression cassettes joining a 5′ regulatory regions (promoters), a coding sequence and a 3′ regulatory regions (terminators) as described hereinbelow and as are known in the art. Any combination of these three sequences can form a functional expression cassette.

The list of terminators includes, but are not limited to that of Th. heterothallica genes encoding for uncharacterized protein G2QF75 (XP_003664349); polyubiquitin homologue (G2QHM8, XP_003664133); uncharacterized protein (G2QIA5, XP_003664731); beta-glucosidase (G2QD93, XP_003662704); elongation factor 1-alpha (G2Q129, XP_003660173); chitinase (G2QDD4, XP_003663544) phosphoglycerate kinase (PGK) (Uniprot G2QLD8), glyceraldehyde 3-phosphate dehydrogenase (GPD) (G2QPQ8), phosphofructokinase (PFK) (G2Q605); or triose phosphate isomerase (TPI) (G2QBR0); actin (ACT) (G2Q7Q5); cbh1 (GenBank AX284115) or β-glucosidase 1 bgl1 (XM_003662656). Exogenous terminators include that of Aspergillus nidulans gpdA terminator.

5′ regulatory regions (promoters) are practically defined as a stretch of up to 2000 base pairs preceding the start codon of the coding sequence of the gene they regulate, provided that the preceding region is non-coding.

3′ regulatory regions (terminators) are practically defined as a stretch of up to 300 base pairs downstream from the end codon of the coding sequence of the gene, provided that the subsequent region is non-coding.

DNA sequences are also assembled to selection marker cassettes, which are expression cassettes where the coding sequence codes for a gene that provides a selective advantage when present in a transformed strain. Such advantage can be utilization of a new carbon or nitrogen source, a resistance to a toxic substance, etc.

Deletion of the proteases disclosed herein can be done as known in the art. In some embodiments, the deletion is performed by transformation of suitable DNA constructs. DNA constructs used for targeted transformation are composed of (a) a suitable vector that allows the maintenance of the DNA construct in a particular host, (b) zero, one or more expression cassettes in any direction, (c) a selection marker cassette in any direction and (d) sequences that are identical to select stretches of the target genomic DNA (also called as targeting arms). These components are placed so, that the two targeting arms encompass any expression cassettes and the selection marker cassette, so that when homologous recombination happens between the targeting arms and the two identical regions in the genomic DNA, the sequence between the targeting arms of the DNA constructs gets inserted into the chromosome, and replaces the sequence originally present on the chromosome. Using this principle, genes can be knocked out from, or inserted into the genome. By placing a sequence downstream of the selection marker cassette, which is identical to the sequence just upstream of the selection marker cassette, it is possible to recycle the marker as known in the art.

The term “regulatory sequences” refer to DNA sequences which control the expression (transcription) of coding sequences, such as promoters, enhancers and terminators.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent or environmental condition). Promoters may also restrict transcription to a certain developmental stage or to a certain morphologically distinct part of the organism. In most cases the exact boundaries of regulatory sequences have not been completely defined, and in some cases, cannot be completely defined, and thus DNA sequences of some variation may have identical promoter activity.

The term “terminator” is directed to another regulatory DNA sequence which regulates transcription termination. A terminator sequence is operably linked to the 3′ terminus of the nucleic acid sequence to be transcribed.

The terms “C1 promoter” and “C1 terminator” indicate promoter and terminator sequences suitable for use in C1, i.e., capable of directing gene expression in C1.

However, as known to the skilled artisan, the choice of promoters and terminators may not be critical, and similar results can be obtained with a variety of promoters and terminators providing similar or identical gene expression.

The term “operably linked” means that a selected nucleic acid sequence is in proximity with a regulatory element (promoter, enhancer and/or terminator) to allow the regulatory element to regulate expression of the selected nucleic acid sequence.

The present invention discloses the production of protein of interest using genetically modified strains of Th. heterothallica C1. As described hereinabove, filamentous fungi of other species sharing endogenous similar pathways of precursor production can be also used.

According to certain embodiments, the polynucleotides of the present invention are designed based on the amino acid sequence of the protein to be produced employing a codon usage of a filamentous fungus. According to certain embodiments, the filamentous fungus belongs to the group Pezizomycotina. According to some embodiments, the filamentous fungus belongs to a group selected from the group consisting of Sordariales, Hypocreales Onygenales, and Eurotiales including genera and species as described in the “definition” section hereinabove. According to certain exemplary embodiments, the fungus is Th. heterothallica. According to these embodiments, the polynucleotides of the present invention are polynucleotides identified in Th. heterothallica or homologs thereto. According to certain currently exemplary embodiments, the fungus is Th. heterothallica C1.

According to certain exemplary embodiments, the Th. heterothallica C1 strain is a derivative of strain UV18-#100.

The DNA constructs or expression vector or plurality of same each comprises regulatory elements controlling the transcription of the polynucleotides within the at least one fungus cell. The regulatory element can be a regulatory element endogenous to the fungus, particularly to Th. heterothallica C1 or exogenous to the fungus.

According to certain embodiments, the regulatory element is selected from the group consisting of a 5′ regulatory element (collectively referred to as promoter), and 3′ regulatory element (collectively referred to as terminator), even though these nucleotide sequences may contain additional regulatory elements not classified as promoter or terminator sequences in the strict sense.

According to certain embodiments, the DNA construct or expression vector comprises at least one promoter operably linked to at least one polynucleotide containing a coding sequence, operably linked to at least one terminator. According to certain embodiments, the promoter is endogenous promoter of the fungus, particularly to Th. heterothallica. According to additional or alternative embodiments, the promoter is heterologous to the fungus, particularly to Th. heterothallica. According to certain embodiments, the terminator is endogenous terminator of the fungus, particularly to Th. heterothallica. According to additional or alternative embodiments, the terminator is heterologous to the fungus, particularly to Th. heterothallica.

According to certain exemplary embodiments, the DNA constructs contain synthetic regulatory elements called as “synthetic expression system” (SES) essentially as described in International (PCT) Application Publication No. WO 2017/144777.

According to certain embodiments, the polynucleotide is stably integrated into at least one chromosomal locus of the at least one cell of the genetically modified fungus. According to certain embodiments, the polynucleotide is stably integrated into a defined site on the fungal chromosomes. According to certain embodiments, the polynucleotide is stably integrated into a random site of the chromosome. According to certain embodiments, the polynucleotide may be incorporated in targeted or random fashion as 1, 2 or more copies to 1, 2 or more chromosomal loci.

According to certain alternative embodiments, the polynucleotide is transiently expressed using extrachromosomal expression vectors as is known to a person skilled in the art.

According to certain embodiments, culturing of the genetically modified fungus in a suitable medium provides for production of protein of interest in an increased amount compared to the amount produced in a corresponding parent fungus cultured under similar conditions.

According to certain exemplary embodiments, the present invention provides a genetically modified Th. heterothallica C1 fungus that enables producing a protein of interest. According to these embodiments, such genetically modified Th. heterothallica C1 fungus comprises at least one cell having reduced expression and/or activity of KEX2 and/or ALP7 and at least one additional protease.

According to certain embodiments, a suitable medium for culturing the genetically modified fungi comprises a carbon source selected from the group consisting of glucose, sucrose, xylose, arabinose, galactose, fructose, lactose, cellobiose, and glycerol. According to some embodiments, the carbon source is provided from waste of ethanol production or other bioproduction from starch, sugar beet and sugar cane such as molasses comprising fermentable sugars, starch, lignocellulosic biomass comprising polymeric carbohydrates such as cellulose and hemicellulose.

According to certain currently exemplary embodiments, the fungus is Th. heterothallica C1. According to certain embodiments, the strain of Th. heterothallica C1 is selected from the group consisting of strain UV18-25, deposit No. VKM F-3631 D; strain NG7C-19, deposit No. VKM F-3633 D; and strain UV13-6, deposit no. VKM F-3632 D. Additional strains that may be used are HC strain UV18-100f deposit No. CBS141147; HC strain UV18-100f deposit No. CBS141143; LC strain W1L #100I deposit No. CBS141153; and LC strain W1L #100I deposit No. CBS141149 and derivatives thereof. Each possibility represents a separate embodiment of the present invention.

According to another aspect, the present invention provides a method for producing a fungus capable of producing an exogenous protein of interest, the method comprising transforming at least one cell of the fungus with at least one polynucleotide encoding to the protein of interest, said at least one cell of the fungus having reduced expression and/or activity of KEX2 and/or ALP7 and at least one additional protease.

According to some embodiments, the method further comprises deleting, inhibiting, or reducing the expression of KEX2 or ALP7. According to some embodiments the method further comprises deleting, inhibiting, or reducing the expression of at least one protease selected from the group consisting of ALP1, PEP4, ALP2, PRT1, SRP1, ALP3, PEP1, MTP2, PEP5, MTP4, PEP6, ALP4.

The terms “reduced expression” or “inhibited expression” of a protein, in particular protease, as described herein are used interchangeably and include, but are not limited to, deleting or disrupting the gene that encodes for the protein.

The terms “reduced activity” or “inhibited activity” of a protein, in particular protease, as described herein are used interchangeably further include posttranslational modifications resulting in reduced or abolished activity of the protein.

Any method as is known in the art for transforming filamentous fungi with polynucleotide encoding for the protein of interest can be used according to the teachings of the present invention.

The fungus and the polynucleotides are as described hereinabove.

According to yet another aspect, the present invention provides a method of producing an exogenous protein, the method comprising culturing the genetically modified fungus, particularly Th. heterothallica C1 fungi of the present invention in a suitable medium; and recovering the protein products.

According to certain embodiments, the method comprises culturing genetically modified fungi as described herein, each expressing a different protein of interest. According to certain embodiments, the fungi express antigens of different coronavirus variants.

According to certain embodiments, the medium comprises a carbon source selected from the group consisting of glucose, sucrose, xylose, arabinose, galactose, fructose, lactose, cellobiose, and glycerol. According to certain embodiments the carbon source is waste obtained from ethanol production or other bioproduction from starch, sugar beet and sugar cane such as molasses comprising fermentable sugars, starch, lignocellulosic biomass comprising polymeric carbohydrates such as cellulose and hemicellulose.

According to some embodiment, the exogenous protein is purified from the fungal growth medium.

According to other embodiments, the exogenous protein is extracted from the fungal mass. Any method as is known in the art for extracting and purifying proteins from vegetative tissues can be used.

According to a further aspect, the present invention provides an exogenous protein produced by the genetically modified fungus, particularly the genetically modified Th. heterothallica C1 of the present invention.

According to some embodiments, the exogenous protein product is a coronavirus antigen. According to some embodiments, the antigen is the full spike protein of coronavirus. According to certain embodiments, the antigen comprises the RBD sequence of the coronavirus spike protein, or fragment thereof. According to certain embodiments, the RBD or fragment thereof is fuses, directly or indirectly, to Spytag. According to certain embodiments, the antigen is attached to a Spy catcher.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Deletion of C1 Alp7 Gene

C1 alp7 protease gene was deleted from the C1 protease deletion lineage strain in which 12 proteases were deleted earlier. The deletion cassette for alp7 was constructed in two parts into two separate plasmids. The marker fragments in these two plasmids overlap with each other, and this region is planned to undergo homologous recombination in C1 between the plasmids at the same time as the 5′ and 3′ flanking region fragments recombine with genomic DNA on both sides of the alp7 gene. The recombination between the selection marker fragments makes the marker gene functional and enables the transformants to grow under selection. The deletion cassette also contains a direct repeat sequence of the 5′ flanking region for the removal of the pyr4 marker. The sequences of the deletion construct plasmids are set forth in SEQ ID NOs: 21 and 22. The 5′ arm plasmid pMYT0936 contained the alp7 5′ flanking region fragment for integration (positions 9-1,025 of SEQ ID NO: 21), and half of pyr4 marker (positions 1,033-2,812 of SEQ ID NO: 21). The 3′ arm plasmid pMYT0937 contained the second half of the pyr4 marker (positions 17-1273 of SEQ ID NO: 22), a direct repeat sequence (positions 1282-1781 of SEQ ID NO: 22), and the alp7 3′ flanking region fragment for integration (positions 1790-2759 of SEQ ID NO: 22).

The alp7 flanking region fragments and the direct repeat were amplified from C1 genomic DNA and cloned into pyr4 marker-containing backbone vectors originating from pSR426 plasmid by Gibson cloning with NEBbuilder™ HiFi DNA Assembly kit (New England Biolabs) according to manufacturer's instructions. Both parts of the deletion construct were excised from the plasmids and co-transformed into the C1 strain DNL146 having 12 deletions of protease genes with protoplast/PEG method as described in Visser, V. J, et al. (Industrial Biotechnology 2011, 7, 214-223).

The transformed colonies growing on the pyr4 selection medium plates were streaked out again on the same selective medium. Identification of correct transformants was carried out by PCR. Mycelium from the transformant streaks was dissolved in 20 mM NaOH and incubated at 100° C. to lyse the cells. 1-2 μl of this solution was used as template for PCR with Phire Plant PCR Kit™ (Thermo Fisher). The oligonucleotide primers used in this PCR are shown in Table 1. The integration of the deletion construct into the alp7 locus was shown by two PCR reactions. Integration at the 5′ end of the gene was shown by a reaction with the primers set forth as SEQ ID NOs: 25 and 26. A 1233 bp fragment was amplified and this showed successful integration to alp7 locus. Integration at the 3′ end of alp7 was shown with the primers set forth as SEQ ID NOs: 27 and 28. A 1748 bp fragment was amplified and this showed successful integration to alp7 locus. The loss of alp7 gene was shown by a reaction with the primers set forth as SEQ ID NOs: 29 and 30. No fragment of 569 bp was amplified and this showed the deletion of alp7 gene.

TABLE 1
Oligonucleotide primers used for showing correct
integration and loss of alp7.

| SEQ ID NO: | Sequence |
|---|---|
| 25 (oMYT2190) | CCTGCATTGCAAGTTCCCAC |
| 26 (oMYT0106) | AGTTTGACAGTGCCCAGAGC |
| 27 (oMYT0027) | AGCCTGGAAGGCCTATCTGG |
| 28 (oMYT0693) | GGTCGGATTGGCTTGGTACA |
| 29 (oMYT0694) | ACCACCGTCAACACGTACAA |
| 30 (oMYT0695) | CAAAGGTCTTGCCACCGATG |
| 31 (oMYT2193) | TTCGTTGCTAACACTCCCCC |
| 32 (oMYT2194) | CTGGTTGATGGCCGAGTTGA |

Transformants positive for both integration PCR reactions and positive for the loss of alp7 orf were further analyzed by quantitative PCR with the primers set forth as SEQ ID NOs: 31 and 32 to demonstrate that the alp7 gene had been completely deleted from the tested transformants. One C1 transformant clone, positive for integration of the deletion cassette into alp7 locus and negative in the qPCR test for presence of alp7 gene, was stored at −80° C. and given the strain number DNL150.

### Example 2: Deletion of C1 Kex2 Gene

C1 kex2 protease gene was deleted from the C1 protease deletion lineage strain in which 12 proteases were deleted earlier. The deletion cassette for kex2 was constructed in two parts into two separate plasmids and it functions upon transformation to C1 in a similar manner as the alp7 deletion cassette (described above). The deletion cassette also contains a direct repeat sequence of the 5′ flanking region for the removal of the pyr4 marker. The sequences of the deletion construct plasmids are set forth in SEQ ID NOs: 23 and 24. The 5′ arm plasmid pMYT0997 contained the kex2 5′ flanking region fragment for integration (positions 9-1,058 of SEQ ID NO: 23), and half of pyr4 marker (positions 1,033-2,812 of SEQ ID NO: 23). The 3′ arm plasmid pMYT0998 contained the second half of the pyr4 marker (positions 17-1273 of SEQ ID NO: 24), a direct repeat sequence (positions 1281-1782 of SEQ ID NO: 24), and the kex2 3′ flanking region fragment for integration (positions 1791-2690 of SEQ ID NO: 24).

The fragments of kex2 flanking regions and the direct repeat were amplified from C1 genomic DNA and cloned into pyr4 marker-containing backbone vectors originating from pSR426 plasmid by Gibson cloning with NEBbuilder™ HiFi DNA Assembly (New England Biolabs) according to manufacturer's instructions. Both parts of the deletion construct were excised from the plasmids and co-transformed into the C1 strain DNL146 having 12 deletions of protease genes as described before in Visser, V. J, et al. (ibid).

The transformed colonies growing on the pyr4 selection medium plates were streaked out again on the same selective medium. Identification of correct transformants was carried out by PCR. Mycelium from the transformant streaks was dissolved in 20 mM NaOH and incubated at 100° C. to lyse the cells. 1-2 μl of this solution was used as template for PCR with Phire Plant PCR Kit™ (Thermo Fisher). The oligonucleotide primers used in this PCR are shown in Table 2. The integration of the deletion construct into the kex2 locus was shown by two PCR reactions. Integration at the 5′ end of the gene was shown by a reaction with the primers set forth as SEQ ID NOs: 33 and 34. A 1187 bp fragment was amplified and this showed successful integration to kex2 locus. Integration at the 3′ end of kex2 was shown with the primers set forth as SEQ ID NOs: 35 and 36. A 1849 bp fragment was amplified and this showed successful integration to kex2 locus. The loss of kex2 gene was shown by a reaction with the primers set forth as SEQ ID NOs: 37 and 38. No fragment of 510 bp was not amplified and this showed the deletion of kex2 gene.

TABLE 2
Oligonucleotide primers used for showing correct
integration and loss of kex2

| SEQ ID NO: | Sequence |
|---|---|
| 33 (oMYT2305) | GGCAGATTATTCCGGACCGT |
| 34 (oMYT0106) | AGTTTGACAGTGCCCAGAGC |
| 35 (oMYT0027) | AGCCTGGAAGGCCTATCTGG |
| 36 (oMYT2306) | TCAACGTGTGGGAGCAGTAC |
| 37 (oMYT2299) | GGGCTCCATCTACGTCTTCG |
| 38 (oMYT2300 | TGGATCCAGGGCGAGTAGAA |
| 39 (oMYT2301) | TGGGCTCGTACGACTTCAAC |
| 40 (oMYT2302) | CGGCGATGTTGGAGTCGTAT |
| 41 (oMYT2303) | CGAGACCGACAAGACCAACA |
| 42 (oMYT2304) | GAAGAGCACGATGAGCACGA |

Transformants positive for both integration PCR reactions and positive for the loss of kex2 ORF were further analyzed by quantitative PCR with the primers set forth as SEQ ID NOs: 39 and 40 and with the primers set forth as SEQ ID NOs: 41 and 42 to 20 demonstrate that the kex2 gene had been completely deleted from the tested transformants. One C1 transformant clone, positive for integration of the deletion cassette into kex2 locus and negative in the qPCR test for presence of kex2 gene, was stored at −80° C. and given the strain number DNL152.

### Example 3: Combining Deletions of C1 Alp7 Gene and C1 Kex2 Gene

The generation of a C1 strain in which both alp7 gene and kex2 gene are deleted, was performed by deleting kex2 gene from DNL150 strain in which alp7 gene and 12 other protease genes were deleted earlier. Prior to the deletion of kex2 gene, the pyr4 marker in the DNL150 strain was removed in order to use the same deletions cassettes for deletion of kex2 gene as described in generation of DNL152 strain above.

The removal of pyr4 selection marker using the deletion cassette described in generation of DNL150 above is based on two features: a) a functional pyr4 gene converts 5-Fluoroorotic acid (5-FOA) into 5-Fluorouracil, a toxic metabolite, thus clones which have lost a functional pyr4 gene are able to grow in the presence of 5-FOA; and b) under 5-FOA selection pressure the direct repeat sequence in the deletion construct enables the clones to remove the pyr4 selection marker by a homologous recombination event between the 5′ flanking region and the direct repeat. Successful recombination loops out the complete pyr4 marker enabling the correct clones to grow in the presence of 5-FOA.

The pyr4 marker removal from DNL150 was carried out according to the following protocol: a small portion of fresh mycelium from a plate was suspended into 0.9% NaCl, 0.025% Tween20 solution. Dilutions of the suspension were prepared. Varying amounts of mycelial suspension were spread onto 5-Fluoroorotic acid (5-FOA) containing plates (medium components of 5-FOA plates: 7 mM KCl, 11 mM KH₂PO₄, 0.1% Glucose, 10 mM Uracil, 10 mM Uridine, 2 mM MgSO₄, 10 mM Proline, Trace element solution (1000×: 174 mM EDTA, 76 mM ZnSO₄·7H₂O, 178 mM H3BO₃, 25 mM MnSO₄·H₂O, 18 mM FeSO₄·7H₂O, 7.1 mM CoCL₂·6H₂O, 6.4 mM CuSO₄·5H₂O, 6.2 mM Na₂MoO₄·2H₂O), 4 mM 5-Fluoroorotic acid, 20 g/l agar granulated, pH 6.0). Plates were incubated at +35° C. until colonies emerged. The colonies growing on the 5-FOA medium plates were streaked out again on the same selective medium. Since growth on 5-FOA selective medium is poor and streaks are not growing as clear streaks, the mycelium from weak streaks were re-streaked on non-selective medium (medium components: 7 mM KCl, 55 mM KH₂PO₄, 1.0% Glucose, 670 mM Sucrose, 0.6% Yeast extract, 35 mM (NH₄)2SO₄, 2 mM MgSO₄, 10 mM Uracil, 10 mM Uridine, Trace element solution (1000×: 174 mM EDTA, 76 mM ZnSO₄·7H₂O, 178 mM H3BO₃, 25 mM MnSO₄·H₂O, 18 mM FeSO₄·7H₂O, 7.1 mM CoCL₂·6H₂O, 6.4 mM CuSO₄·5H₂O, 6.2 mM Na₂MoO₄·2H₂O), 16 g/l agar granulated, pH 6.5) to obtain good growth. The streaks growing efficiently on non-selective medium plates were streaked on pyr4 selective medium plates without uracil and uridine for phenotype testing. In phenotype testing, clones in which pyr4 removal is successful are unable to grow on medium without uracil and uridine supplementation (medium components: 7 mM KCl, 11 mM KH₂PO₄, 1.0% Glucose, 670 mM Sucrose, 35 mM (NH₄)2SO₄, 2 mM MgSO₄, Trace element solution (1000×: 174 mM EDTA, 76 mM ZnSO₄·7H₂O, 178 mM H3BO₃, 25 mM MnSO₄·H₂O, 18 mM FeSO₄·7H₂O, 7.1 mM CoCL₂·6H₂O, 6.4 mM CuSO₄·5H₂O, 6.2 mM Na₂MoO₄·2H₂O), 15 g/l agar granulated, pH 6.5). The clones not growing in phenotype testing plates were analyzed for removal of pyr4 by quantitative PCR with the primers set forth as Sequence No: 43 and 44. The oligonucleotide primers used in the qPCR reactions are shown in Table 3.

TABLE 3
Oligonucleotide primers used in quantitative PCR
for loss of pyr4

| SEQ ID NO: | Sequence |
|---|---|
| 43 (oMYT1292) | TTGGTAAGACGGTGCAGATG |
| 44 (oMYT1293) | GTAGTTGATGCGTTCCTTCCA |

One DNL150 pyr4 loopout clone unable to grow in the phenotype testing and showing negative result for pyr4 gene in quantitative PCR, was stored at −80° C. and given the strain number DNL151.

Kex2 protease was deleted from the C1 strain DNL151 with same deletion cassette and transformation method as described above in the generation of DNL152. Identification of correct integration and deletion of kex2 by PCR reaction were performed as described above in the generation of DNL152. One C1 transformant clone, positive for integration of the deletion cassette into kex2 locus and negative in the qPCR test for presence of kex2 gene, was stored at −80° C. and given the strain number DNL155 (Δalp1Δalp2Δpep4Δprt1Δsrp1Δalp3Δpep1Δmtp2Δpep5Δmtp4Δpep6Δ alp4Δalp7Δkex2).

### Example 4: Expression of SARS-CoV-2 RBD in Protease Deficient C1 Strains

Receptor binding domain (RBD) of SARS-CoV-2 spike protein was expressed in protease deficient C1 strains. The first construct contained a sequence coding for a C1 endogenous CBH1 signal sequence, the residues 333-527 of the Spike protein from SARS-CoV-2, a Gly-Ser-linker and the C-tag flanked by recombination sequences to the C1 expression vector and MssI restriction enzyme sites. The fragment was synthetized by GenScript (USA) and is set forth as SEQ ID NO: 45 (RBD-C-tag amino acid sequence, including a signal sequence and Gly/Ser linker between RBD and the C-tag). The codon usage of the gene was optimized for expression in Thermothelomyces heterothallica. The synthetized fragment was amplified by PCR from the GenScript plasmid and cloned by Gibson Assembly (NEBuilder® HiFi DNA Assembly Cloning Kit, New England Biolabs) method into the PacI site of the C1 expression vector pMYT1055 under endogenous C1 bgl8 promoter and C1 chi1 terminator. The correct sequence of the construct was confirmed by sequencing the fragment inserted into the plasmid. A plasmid of correct sequence was given the plasmid number pMYT1142 (SEQ ID NO: 46). A second construct contained in addition to the same sequences as in pMYT1142, a Gly-Ser-linker and a Spytag between the RBD domain and the C-terminal Gly-Ser linker and the C-tag. This sequence is set forth as SEQ ID NO: 47 (RBD-Spytag-C-tag amino acid sequence, including a signal sequence and Gly/Ser linker between the Spytag and the C-tag). The second construct was constructed into the pMYT1055 expression vector in a similar manner as pMYT1142, and a plasmid of correct sequence was given the plasmid number pMYT1143 (SEQ ID NO: 48).

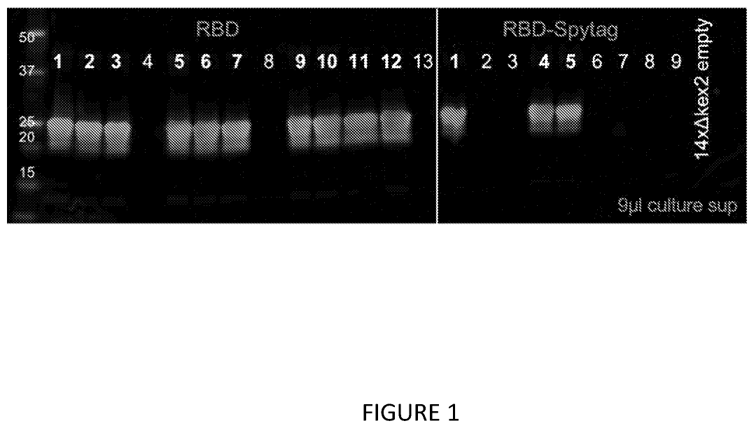

Expression vector pMYT1142 and a mock vector partner pMYT1140, which is needed for completion of the hygromycin resistance marker gene and integration to the bgl8 locus, were digested with MssI and co-transformed to the DNL155 strain from which fourteen protease genes have been deleted. The transformation was done with protoplast/PEG method (Visser, V. J, et al., ibid) and transformants were selected for nia1+phenotype and hygromycin resistance. Transformants were streaked onto selective medium plates and inoculated from the streaks to liquid cultures in 24-well plates. The medium components were (in g/L) glucose 5, yeast extract 1, (NH₄)2SO₄ 4.6, MgSO₄·7H₂O 0.49, KH₂PO₄ 7.48, and (in mg/L) EDTA 45, ZnSO₄·7H₂O 19.8, MnSO₄·4H₂O 3.87, CoCl₂·6H₂O 1.44, CuSO₄·5H₂O 1.44, Na₂MoO₄·2H₂O 1.35, FeSO₄·7H₂O 4.5, H₃BO₄ 9.9, D-biotin 0.004, 50 U/ml Penicillin and 0.05 mg Streptomycin. The 24-well plates were incubated at 35° C. with 800 RPM shaking for four days. Culture supernatants were collected and analysed by Western blotting performed with standard methods with the primary detection agent Capture Select Biotin Anti-C-tag conjugate (ThermoFisher) and the secondary agent IRDye 800CW Stretavidin (Li-Cor). Western analysis (FIG. 1) showed that a strong signal of the expected size for a number of RBD-C-tag and RBD-Spytag-C-tag transformants was detected, showing that both of these proteins were produced in C1.

Transformants producing the RBD-C-tag protein were purified by single colony plating, and a purified clone was verified by PCR for correct integration of the expression cassette and by qPCR for clone purity. One verified transformant producing RBD-C-tag was stored at −80° C. and given the strain number M4169.

Expression vector pMYT1143 carrying the RBD-Spytag-C-tag version was co-transformed with the mock vector partner pMYT1140 and transformants were analysed from 24-well plate cultures (FIG. 1) and purified by single colony plating in the same manner as described above for pMYT1142. After PCR verification, one C1 transformant clone producing RBD-Spytag-C-tag was stored at −80° C. and given the strain number M4173.

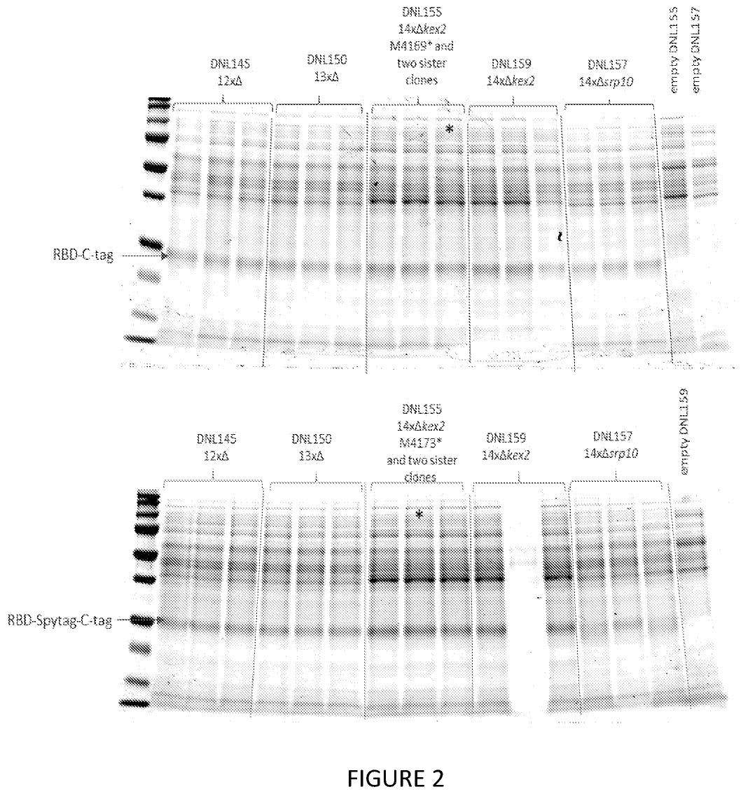

Plasmids pMYT1142 and pMYT1143 were transformed also to other C1 protease deficient strains than DNL155 to compare the production levels in the different protease deletion strains. The protease gene deletions in these strains are listed in Table 4. The RBD-producing plasmids pMYT1142 and pMYT1143 were transformed to four other protease deficient strains: 1) DNL145 strain in which 12 proteases have been deleted, 2) DNL150 in which 13 proteases have been deleted, 3) DNL159 which is a parallel clone of DNL155 and 4) DNL157 in which 14 proteases are deleted but kex2 gene is intact. Transformation, analysis of transformants, single colony purification and PCR analysis were performed in a same manner as described above for generating the strains M4169 and M4173. Several verified production strains were obtained for both production of RBD-C-tag and RBD-Spytag-C-tag in all four protease deficient strains. Three parallel transformants from these new production strains in DNL145, DNL150, DNL157 and DNL159 together with M4169 and M4173 and two other parallel clones of both of these strains were cultivated in liquid medium in 24-well plates at 35° C. with 800 RPM shaking for four days. Culture supernatants were collected and analyzed in Coomassie stained SDS gels with methods known in the art. Highest production of RBD proteins was observed in DNL155 and DNL159 strains from which kex2 is deleted (FIG. 2).

TABLE 4
C1 protease deleted in C1 protease deficient strains

| Strain | C1 protease genes deleted |
|---|---|
| DNL145 | alp1 alp2 pep4 prt1 srp1 alp3 pep1 mtp2 pep5 mtp4 pep6 alp4 |
| DNL150 | alp1 alp2 pep4 prt1 srp1 alp3 pep1 mtp2 pep5 mtp4 pep6 alp4 |
| | alp7 |
| DNL155 | alp1 alp2 pep4 prt1 srp1 alp3 pep1 mtp2 pep5 mtp4 pep6 alp4 |
| | alp7 kex2 |
| DNL159 | alp1 alp2 pep4 prt1 srp1 alp3 pep1 mtp2 pep5 mtp4 pep6 alp4 |
| | alp7 kex2 |
| DNL157 | alp1 alp2 pep4 prt1 srp1 alp3 pep1 mtp2 pep5 mtp4 pep6 alp4 |
| | alp7 srp10 |

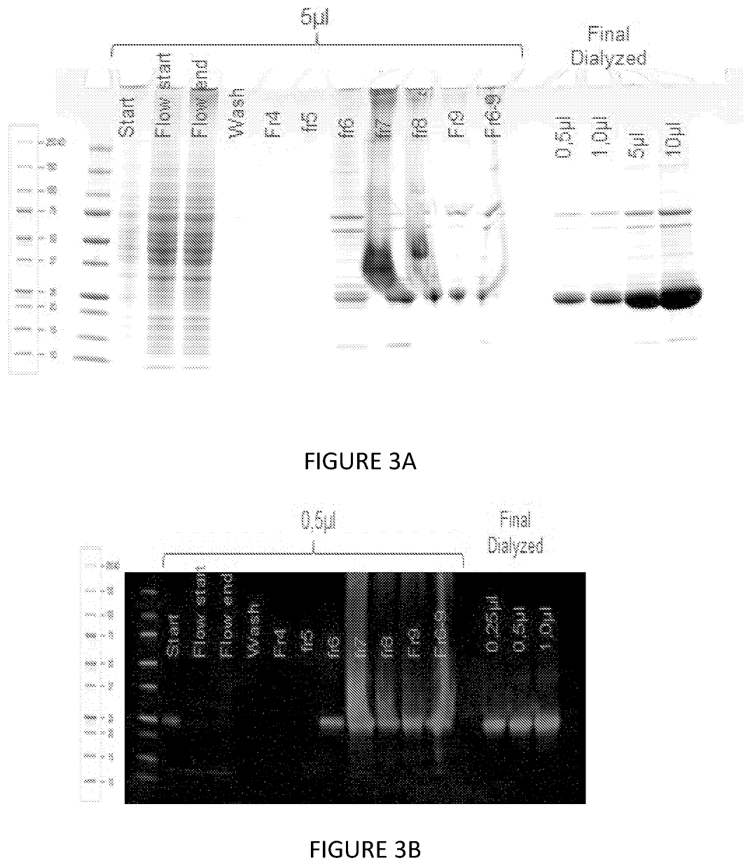

The C1 strain M4169 producing RBD-C-tag protein was cultivated in 2 L bioreactor in a fed-batch process in a medium with yeast extract as an organic nitrogen source and glucose as a carbon source. The culture was performed at 38° C. for five days. After ending the cultivation, mycelia were removed by centrifugation at 4000 g for 20 minutes, phenylmethylsulfonyl fluoride was added in 1-2 mM concentration to inhibit protease activity in the obtained liquid culture supernatant and the supernatant was stored at −80° C. For RBD purification by C-tag affinity chromatography, 100 ml of liquid culture was thawed on ice, and after thawing the sample was clarified by centrifugation 3×20 min 20000 g at +4° C. followed by filtration through a 0.4504 filter. 90 ml of the clear supernatant was diluted with 1×PBS (12 mM Na₂HPO₄*2 H₂O, 3 mM NaH₂PO₄*H₂O, 150 mM NaCl pH 7.3) to final volume of 200 ml. The C-tag affinity purification was performed with 10 ml column of packed CaptureSelect C-tagXL resin (Thermo Fisher) attached to ÄKTA Start protein purification system (Cytiva) and operated with a flow rate of 2.5 ml/min. Column was first equilibrated with 5 column volumes (CV) of 1×PBS prior loading the sample. After sample loading, the column was washed with 15CV of 1×PBS and then eluted with one-step gradient of 5CV of 20 mM Tris-HCl, 2M MgCl₂, 1 mM EDTA pH7.5 with fraction volume of 3 ml. The quantity of the eluted RBD was quantified by integrating the UV trace of the elution peak with the Unicorn 1.0 software included in the ÄKTA Start system. The extinction coefficient of 1.498 was used in calculating RBD-C-tag amount and 1.450 for calculating RBD-Spytag-C-tag amount. After elution, the column was regenerated with 5CV of 0.1M glycine pH 2.3 and washed with 1×PBS till pH7.3 was reached. Elution fractions containing the protein were pooled for dialysis step to exchange the elution buffer to 1×PBS buffer. Pooled fractions were packed in a 12 ml dialysis cassette and the dialysis cassette was dialyzed in 1.51 in 1×PBS for 1 h at +4° C. with stirring on a magnetic stirrer. 1×PBS was changed to fresh buffer after 1 h and dialysis was continued for 2 h in same conditions. Finally, fresh 1×PBS was changed and dialysis was continued overnight. Concentration of dialyzed RBD was determined with the Nanodrop spectrophotometer measuring absorbance at 280 nm and using extinction coefficients 1.498 for RBD-C-tag and 1.450 for RBD-Spytag-C-tag. Aliquots of RBD preparates were stored at −80° C. Affinity purification of RBD-C-tag from M4169 fermentation is shown as an example in FIG. 3A-3B. SARS-CoV-2 Spike RBD Antibody, Rabbit polyclonal antiserum (SinoBiologicals) and Goat anti-rabbit IRDye 680RD (Li-Cor) were used in Western detection.

### Example 5: Expression and Stability of Proteins in Different Strains of Thermothelomyces heterothallica C1

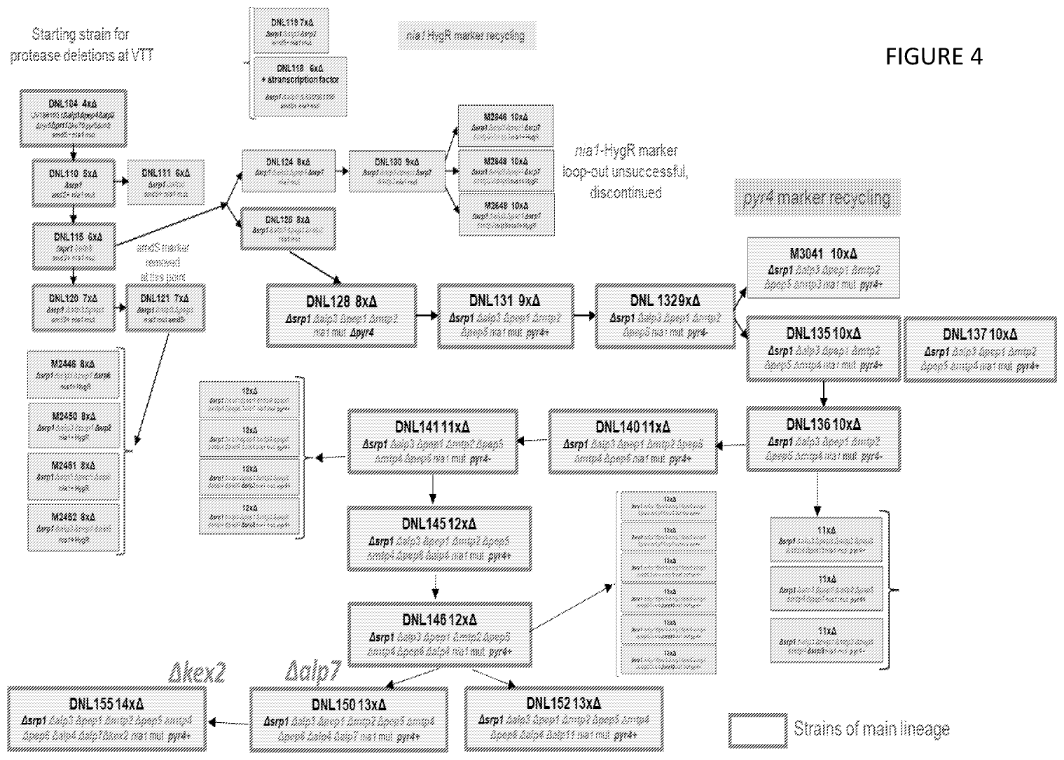

Different strains of Thermothelomyces heterothallica C1, are shown in FIG. 4.

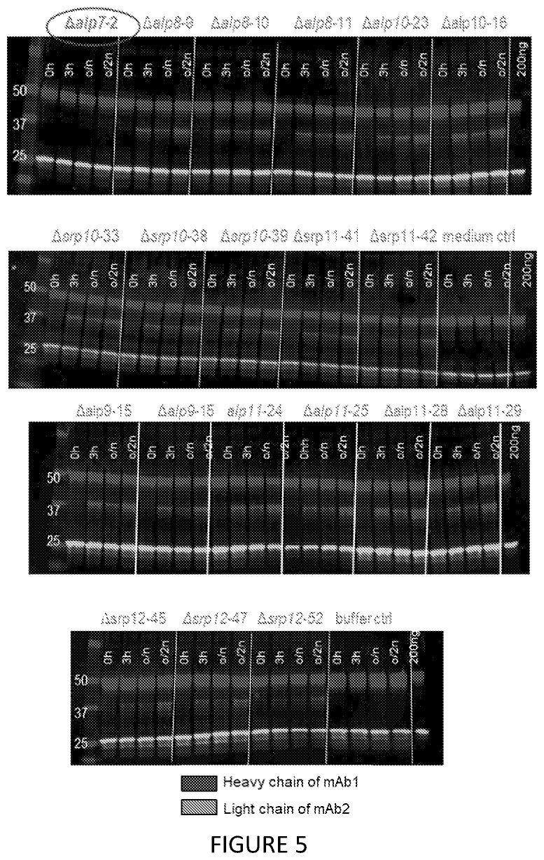

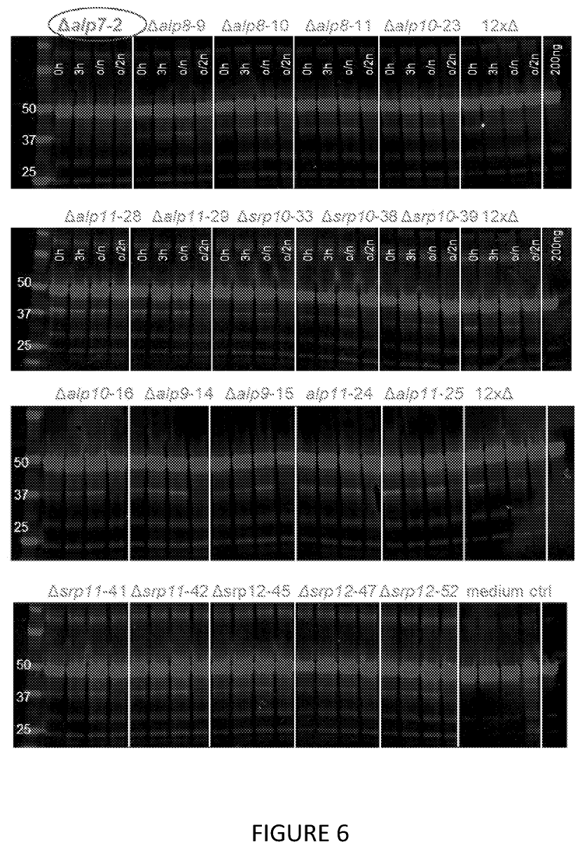

Spiking experiments were used to examine the stability of proteins and antibodies. Target proteins added and incubated in the culture supernatant of the fungal strains. Samples were taken at different time points and analyzed using Western blots. As shown in FIGS. 5 and 6, the deletion of ALP7 had a positive effect on the stability of antibodies.

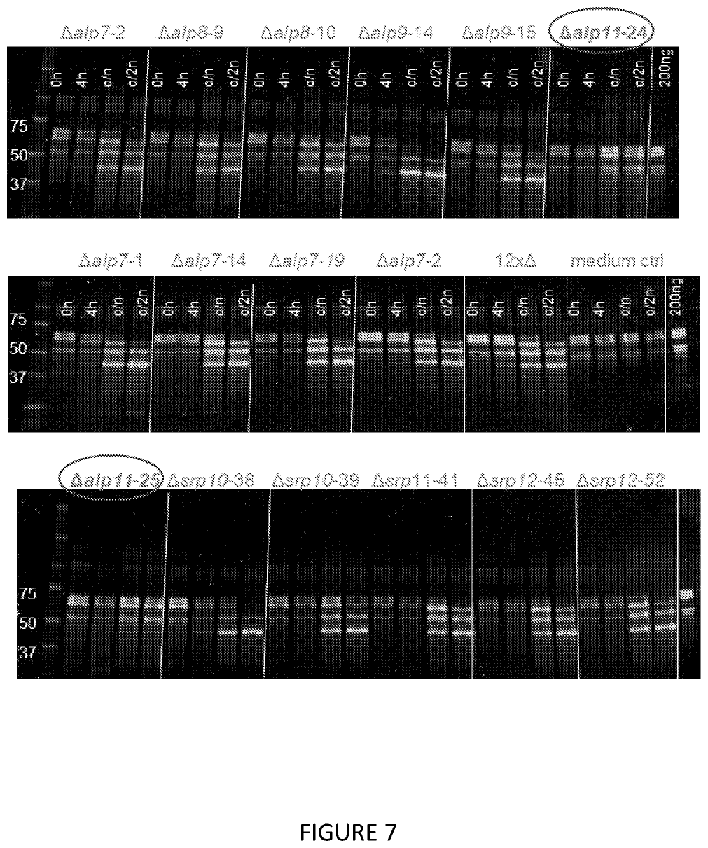

FIG. 7 shows spiking experiments with fibrinogen. Improved stability was found in KEX2 deficient strain.

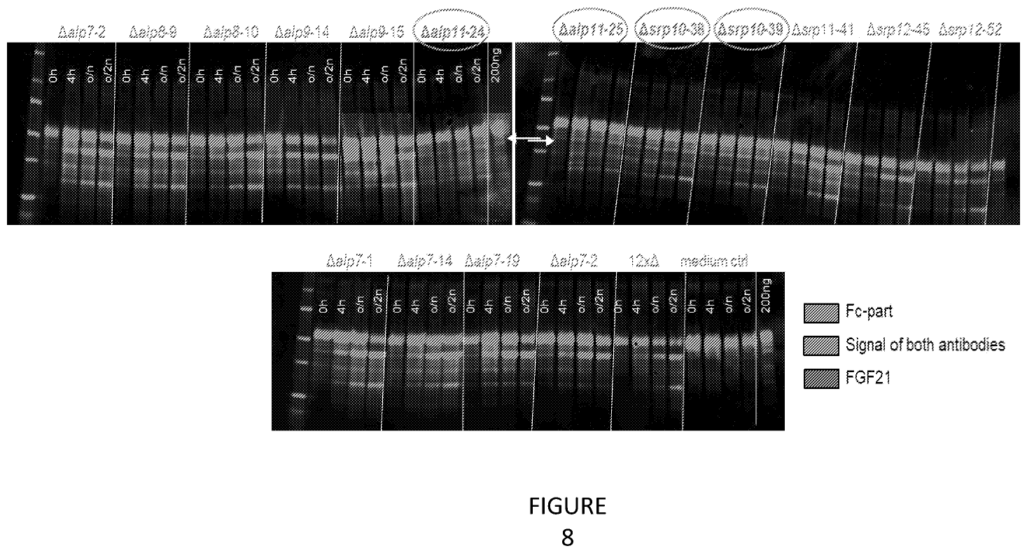

FIG. 8 shows spiking experiments with Fc-FGF21. Improved stability was found in KEX2 and SRP10 deficient strains.

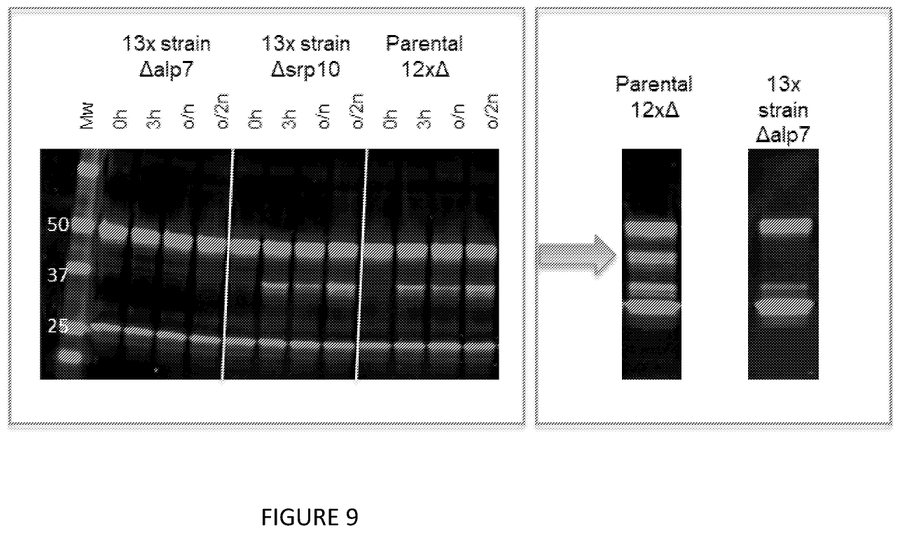

FIG. 9 shows spiking experiment and expression of mAbs in protease deficient strains. Improved stability and protein amounts were found in 13×ALP7 deficient strain compared to 12× and 13×SRP10 proteases deficient strains. When the same mAb was expressed in 13×ALP7 protease deletion strain, much more intact mAb was produced.

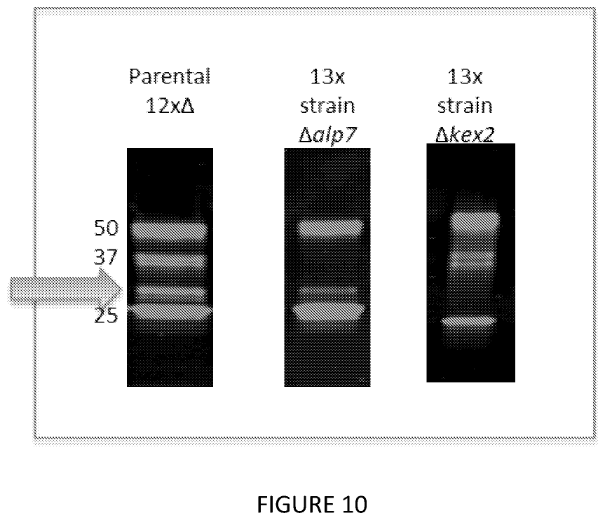

FIG. 10 shows the expression of mAbs in 13× protease deletion strains with either kex2 or alp7 deletion. The 27 kDa degradation fragment (marked with an arrow) was not formed in the KEX2 deletion strain as compared to 12× parental strain. In addition, the 37 kDa degradation fragments were not produced in the 13×ALP7 deficient strain as compared to the 12× protease deficient parental strains.

### Example 6: Expression of RVFV in 14× Proteases Deficient Strains

The vaccine antigen protein from rift valley fever virus was expressed as a fusion protein with Spycatcher domain from the same expression vector in a 13× protease deletion strain DNL150 and in the 14×protease deletion strain DNL155 having kex2 deletion.

The strains transformed with the RVFV antigen expression vector were grown in 24-well plates and production of the antigen was analyzed with Western blotting with an antibody against RVFV antigen.

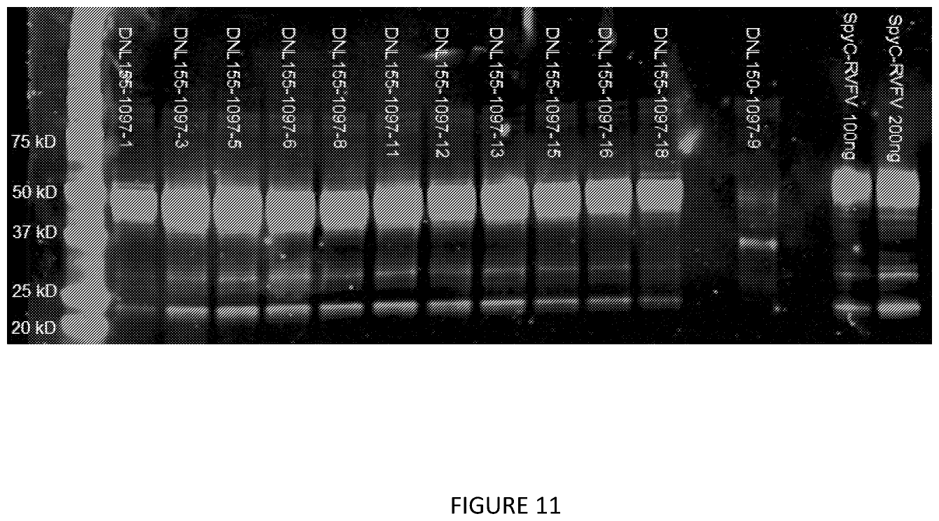

As shown in FIG. 11, the transformants of the 14×protease deficient strain DNL155 showed high expression of RVFV. The expression level was much higher than in the 13× protease deficient strain (DNL150).

### Example 7: Expression and Functionality of RBD-Spytag in 14× Proteases Deficient Strains

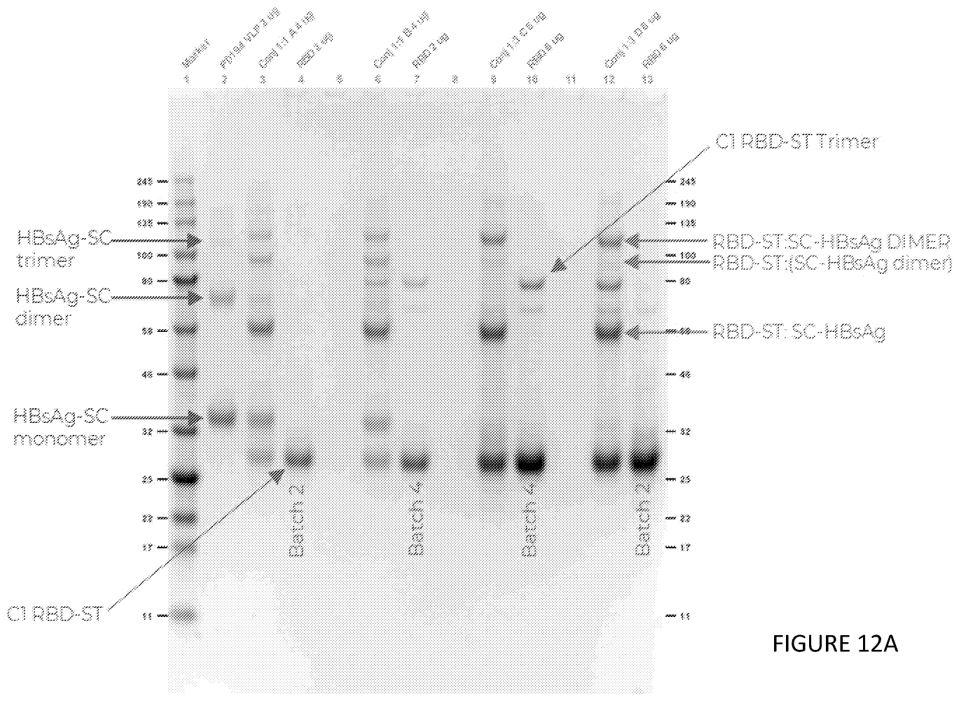

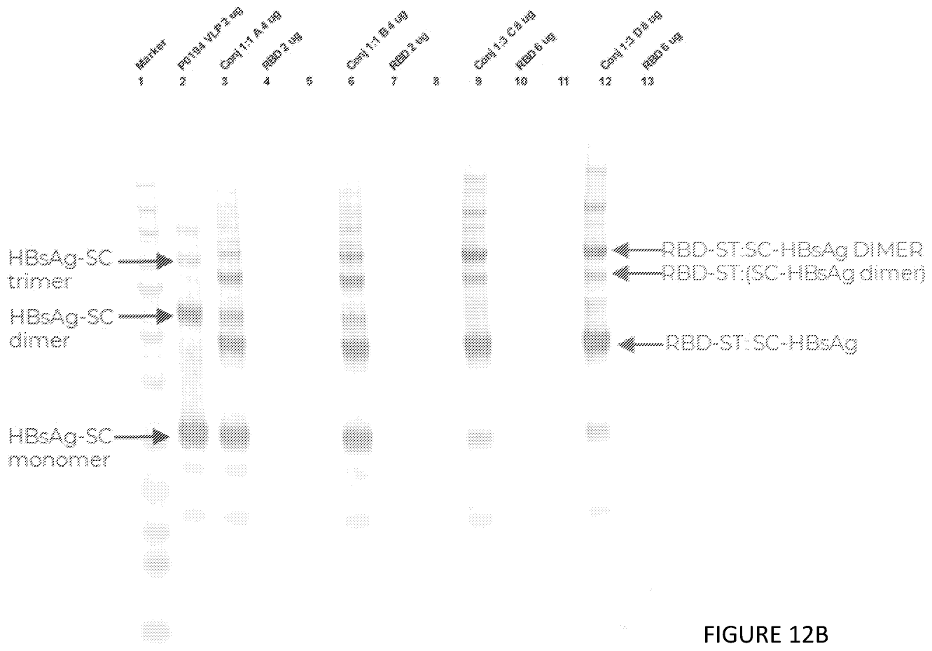

The structural formation of receptor binding domain of SARS-CoV-2 spike protein fused to Spytag in 14×proteases deficient strain of Thermothelomyces heterothallica C1 is presented in FIGS. 12A-12B. The protein was conjugated to SpyCatcher recombinant hepatitis B surface antigen (HBsAg)-virus-like particles (VLP) vaccine to examine the possibility of using the produced protein as a vaccine. Two batches of C1 RBD-Spytag (#2 and #4) were examined. The stability of the proteins and conjugates were examined in an SDS-PAGE gel and later analyzed by Western blot using mouse anti-HBsAg antibody (1^{st} Ab) and goat anti-mouse IgG-Ap (2^{nd} Ab). As shown in FIGS. 12A-12B, the RBD-Spytag was efficiently conjugated to the SpyCatcher HBsAg VLP. Importantly, the RBD proteins with or without the conjugated SpyCatcher were capable of generating dimers/trimers. The dimerization and trimerization of the recombinant RBD simulates the natural structure of the coronavirus RBS and is expected to generate an efficient vaccine.

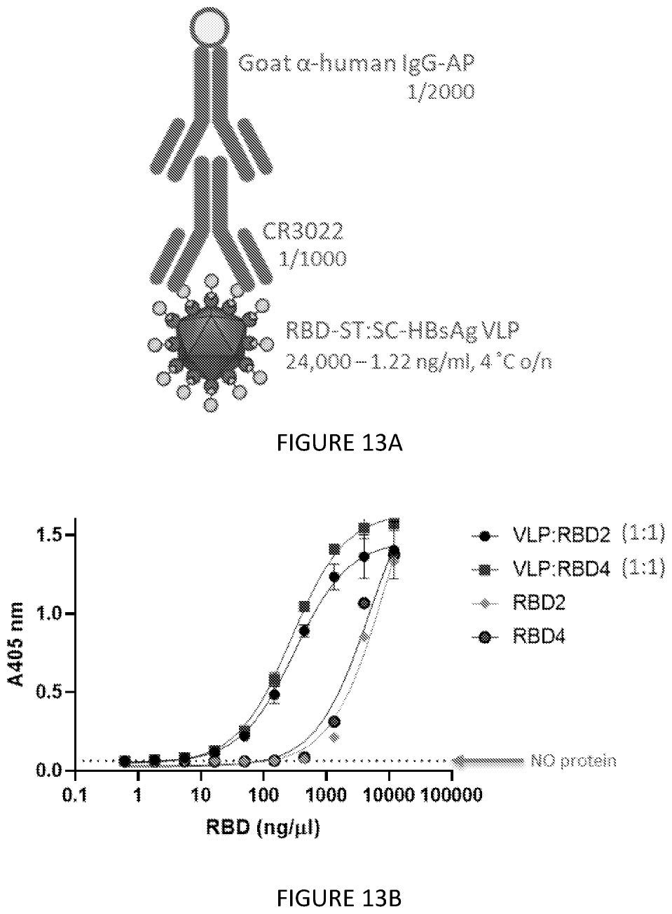

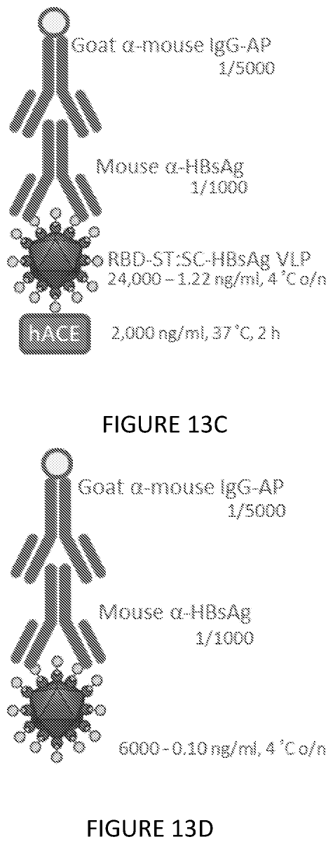

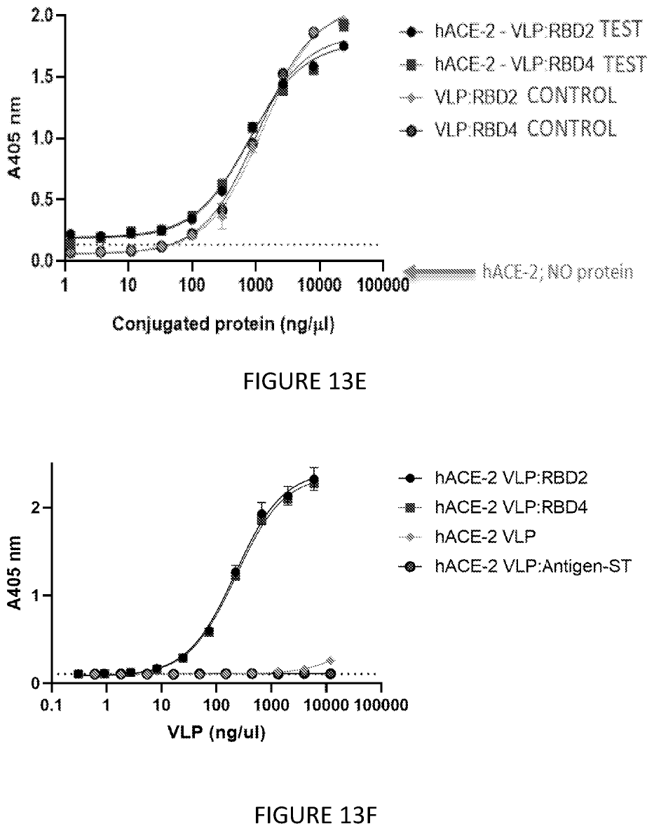

Next, the binding of the RBD-Spytag to human ACE-2 protein was examined using the CR3022 antibody. As shown in FIGS. 13A-13F, the CR3022 antibody is capable of binding to RBD presented on the VLC particle. In addition, an indirect ELISA was used to show that the conjugated RBD binds hACE-2 and not the VLC particle. Together, the results show that the produced RBD fused to Spytag is correctly assembled, presented on the VLC particle and thus, may be used as a vaccine.

### Example 8: Production of Fc Fusion Proteins of the SARS-CoV-2 Receptor Binding Domain in C1

Production of two potential coronavirus SARS-CoV-2 vaccine proteins, where the receptor binding domain (RBD) of the SARS-CoV-2 S2 spike protein was fused either N-terminally or C-terminally with IgG1 antibody Fc domain, was done in C1. DNA fragments encoding a 40 bp overlap with the C1 bgl8 promoter, the C1 CBH1 signal sequence, coding region of either the RBD-Fc or Fc-RBD amino acid sequence (shown as SEQ ID NOs: 49 and 51; said sequences include a signal sequence and a linker between RBD and Fc), a stop codon and an overlap with either the bgl8 or chi1 terminator of C1. The protein coding regions of the DNA fragments are shown as SEQ ID NOs: 50 and 52. The DNA fragments with overlap to the chi1 terminator were cloned into the 5′ arm of the expression construct (plasmid pMYT1055), and the fragments with overlap to the bgl8 terminator were cloned into the 3′ arm of the expression construct (plasmid pMYT1056). Cloning was done with Gibson assembly method with the NEBuilder™ HiFi DNA Assembly kit (New England Biolabs) according to manufacturer's instructions. The resulting expression plasmids were designated pMYT1302 (RBD-Fc 5′ arm), pMYT1303 (RBD-Fc 3′ arm), pMYT1304 (Fc-RBD 5′ arm) and pMYT1305 (Fc-RBD 3′ arm).

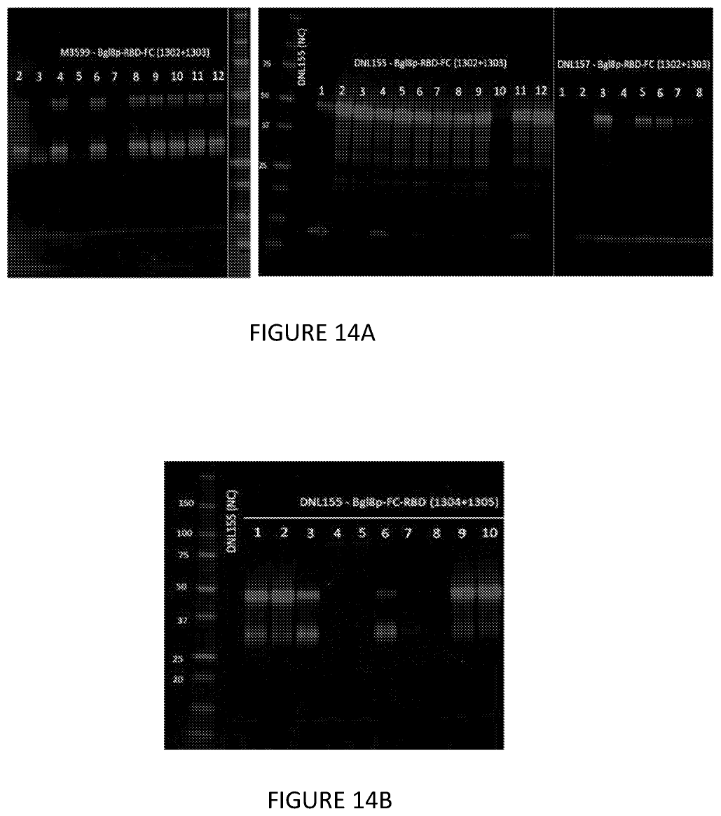

In order to construct RBD-Fc producing C1 strains, the expression plasmids pMYT1302 and pMYT1303 were transformed together into three different C1 strains: DNL155 (Δalp1Δalp2Δpep4Δprt1Δsrp1Δalp3 Δpep1 Δmtp2Δpep5Δmtp4Δpep6Δalp4Δalp7Δkex2), DNL157 (Δalp1Δalp2Δpep4Δprt1Δsrp1Δalp3Δpep1 Δmtp2Δpep5Δmtp4Δpep6Δalp4Δalp7Δsrp10) and a glycoengineered strain having 10 protease deletions, M3599 (Δalp1Δalp2Δpep4Δprt1Δsrp1Δalp3Δpep1Δmtp2Δalp6Δsrp7). Upon transformation the 5′ and 3′ arms of the expression construct integrate to the bgl8 locus and the hygromycin resistance gene overlapping fragments in the two arms recombine with each other to form the final expression construct with two expression cassettes in the bgl8 locus. The transformations were done as described in Visser, V. J, et al. (ibid). Transformants were selected for hygromycin resistance and screened with 24-well plate cultures and Western blotting for production of the RBD-Fc protein. Western analysis was done with standard methodology with 1:10 000 dilution of Anti-human IgG F(c) Goat Polyclonal Antibody-IRDye700DX conjugate (Licor). Signal detection was done with the Licor Odyssey fluorometer device. The results show that only a small minority of RBD-Fc produced in the M3599 strain with 10 protease deletions is of full length (calculated molecular weight 49.4 kDa). In the M155 and M157 strains most of the RBD-Fc is not degraded by proteases and is produced as intact product (FIG. 14A). These strains have alp7 (DNL157) or both alp7 and kex2 (DNL155) protease deletions. Production level in DNL155 is significantly higher than in DNL157. In conclusion, alp7 and kex2 deletions have a beneficial effect on RBD-Fc production.

In order to generate a strain expressing the Fc-RBD fusion protein, plasmids pMYT1304 and pMYT1305 were transformed together into the DNL155 strain as described above for the RBD-Fc production strain construction. Transformants were analyzed for Fc-RBD by Western blotting from 24-well plate cultures as described above (FIG. 14B). Several transformants producing good levels of the Fc-RBD protein were detected. A great majority of the product was intact.

### Example 9: Vaccination of Mice with SARS-CoV-2 RBD Antigen

The produced SARS-CoV-2 spike protein of example 4 was tested for use as a vaccine. The SARS-CoV-2 RBD antigen was injected to K18 hACE2 transgenic mice. Two groups of transgenic mice were vaccinated with 20 μg of RBD formulated with Alhydrogel. The prime vaccination was done on day 1 (‘prime’) and day 21 (‘boost’). At day 42, the mice were challenged with 2000 PFU of SARS-CoV-2. Serum studies revealed that the antigen produced high titers of neutralizing antibodies. Two days following the challenge with SARS, all control mice died, while 13 out of 14 vaccinated mice survived with almost no weight loss.

### Example 10: Expression of αMHCII-Cal07 Recombinant Antigen in Protease Deficient C1 Strains

Recombinant antigen αMHCII-Cal07 consisting of MHCII-targeting domain and HA antigen of influenza strain A/California/07/2009 (subtype H1N1) was expressed in protease deficient C1 strains. The expression construct contained a sequence coding for a C1 endogenous CBH1 signal sequence, a MHCII-specific targeting unit, a 20-aa linker, the residues 18-541 of HA protein derived from the influenza strain A/California/07/2009 and the C-tag flanked by recombination sequences to a C1 expression vector and MssI restriction enzyme recognition sites. The fragment was synthetized by GenScript (USA). The codon usage of the gene was optimized for expression in Thermothelomyces heterothallicus. The synthetized fragment was released from the GenScript plasmid by digestion with the restriction enzyme MssI and cloned by Gibson Assembly (NEBuilder® HiFi DNA Assembly Cloning Kit, New England Biolabs) method into the PacI site of the C1 expression vector pMYT1055 under endogenous C1 bgl8 promoter and C1 chi1 terminator. The correct sequence of the construct was confirmed by sequencing the fragment inserted into the plasmid. A plasmid of correct sequence was given the plasmid number pMYT1242.

In a second instance, the synthesized fragment was amplified by PCR from the GenScript plasmid and cloned by Gibson Assembly method into the PacI site of the C1 expression vectors pMYT0987 under synthetic AnSES promoter and endogenous C1 chi1 terminator. The correct sequence of the construct was confirmed by sequencing the fragment inserted into the plasmid. Plasmid of correct sequence was assigned the plasmid number pMYT1243.

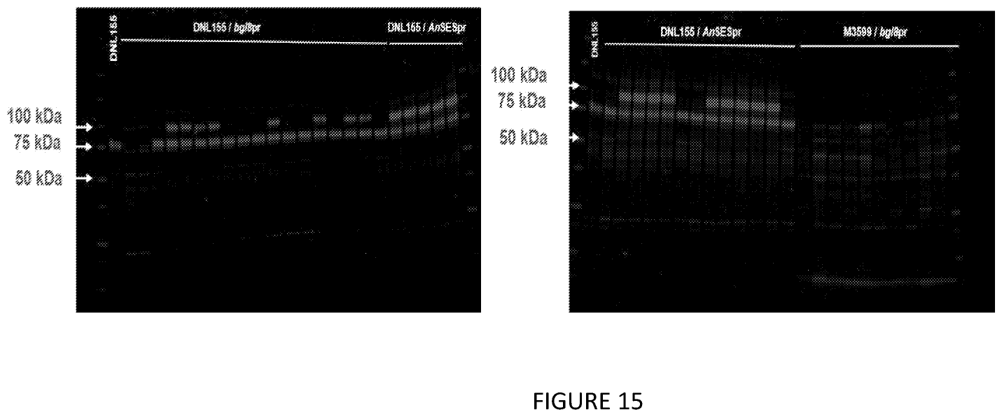

Expression vector pMYT1242 and a mock vector partner pMYT1140, which is needed for completion of the hygromycin resistance marker gene and integration to the bgl8 locus, were digested with MssI and co-transformed to the DNL155 strain from which fourteen protease genes have been deleted, and to the M3599 strain from which ten protease genes have been deleted. Proteases deleted in the above-mentioned strains are listed in the Table 5. The transformation was done with protoplast/PEG method Visser, V. J, et al. (ibid) and transformants were selected for nia1+phenotype and hygromycin resistance. Transformants were streaked onto selective medium plates and inoculated from the streaks to liquid cultures in 24-well plates. The medium components were (in g/L) glucose 5, yeast extract 1, (NH₄)₂SO₄ 4.6, MgSO₄·7H₂O 0.49, KH₂PO₄ 7.48, and (in mg/L) EDTA 45, ZnSO₄·7H₂O 19.8, MnSO₄·4H₂O 3.87, CoCl₂·6H₂O 1.44, CuSO₄·5H₂O 1.44, Na₂MoO₄·2H₂O 1.35, FeSO₄·7H₂O 4.5, H₃BO₄ 9.9, D-biotin 0.004, 50 U/ml Penicillin and 0.05 mg Streptomycin. The 24-well plates were incubated at 35° C. with 800 RPM shaking for four days. Culture supernatants were collected and analysed by Western blotting performed with standard methods with the primary detection agent Capture Select Biotin Anti-C-tag conjugate (ThermoFisher) and the secondary agent IRDye 680RD Streptavidin (Li-Cor). Western analysis (FIG. 15) showed a strong signal of the expected size (87 kDa) for a number of αMHCII-Cal07 transformants derived from DNL155 strain, confirming that the protein was produced in C1. However, no products of expected size could be detected in any of transformants derived from M3599. The additional proteases present in M3599-derived transformants as compared to DNL155-derived transformants had caused proteolytic degradation of the product.

Transformants producing αMHCII-Cal07 protein were purified by single colony plating, and a purified clone was verified by PCR for correct integration of the expression cassette and by qPCR for clone purity. One verified transformant producing αMHCII-Cal07 was stored as a glycerol stock at −80° C. and given the strain number M4540.

C1 strains DNL155 was further co-transformed with the MssI-digested expression vector pMYT1243 carrying the αMHCII-Cal07 construct controlled by the synthetic AnSES promoter together with the mock vector partner pMYT1141, and transformants were analysed from 24-well plate cultures (FIG. 15) and purified by single colony plating in the same manner as described above for pMYT1242. After PCR verification, one C1 transformant clone producing αMHCII-Cal07 was stored at −80° C. and given the strain number M4543.

Additionally, C1 strain M4621, in which fourteen protease gene deletions and deletion of alg3 gene encoding dolichol-P-Man dependent alpha(1-3)mannosyltransferase, was co-transformed with the MssI-digested expression vector pMYT1243 and the mock vector pMYT1141 in the same way as described above for DNL155. Deletion of alg3 gene causes a change in the structure of N-glycans attached to glycoproteins, resulting in a shift to smaller N-glycan species with less mannose residues. Transformants obtained after this transformation were cultivated in liquid medium in 24-well plates at 35° C. with 800 RPM shaking for four days. Culture supernatants were collected and analyzed by Western blotting performed with standard methods with the primary detection agents Capture Select Biotin Anti-C-tag conjugate (ThermoFisher) and murine monoclonal antibody 29E3 raised against HA antigen of the influenza strain A/California/07/2009 (Manicassamy et al., 2010; PLoS Pathog 6(1): e1000745. doi:10.1371/journal.ppat.1000745) and secondary agents IRDye 680RD Streptavidin (Li-Cor) and IRDye 800CW goat anti-mouse IgG secondary antibody (Li-Cor). Western analysis showed the presence of a signal of the expected size (87 kDa) for a number of transformants, confirming that the protein was produced in M4621-derived transformants (data not shown).

TABLE 5
C1 protease deleted in C1 protease deficient strains

| Strain | C1 protease genes deleted |
|---|---|
| M3599 | alp1 alp2 pep4 prt1 srp1 alp3 pep1 mtp2 alp6 srp7 |
| DNL155 | alp1 alp2 pep4 prt1 srp1 alp3 pep1 mtp2 pep5 mtp4 pep6 alp4 |
| | alp7 kex2 |
| M4621 | alp1 alp2 pep4 prt1 srp1 alp3 pep1 mtp2 pep5 mtp4 pep6 alp4 |
| | alp7 kex2 |

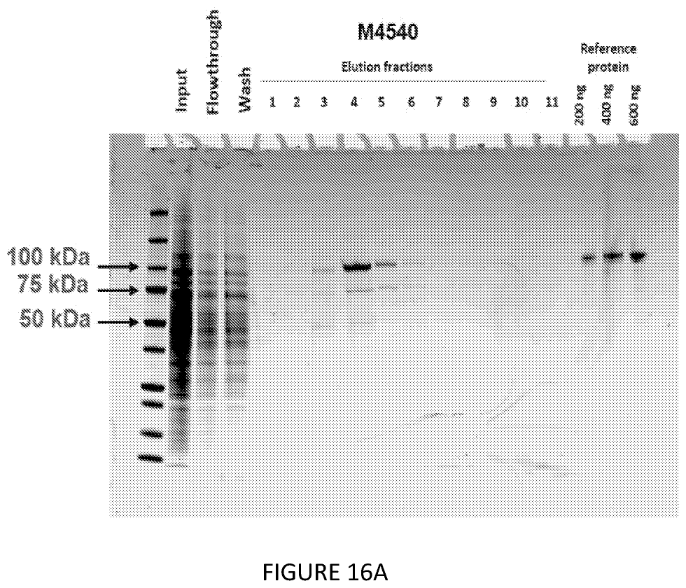

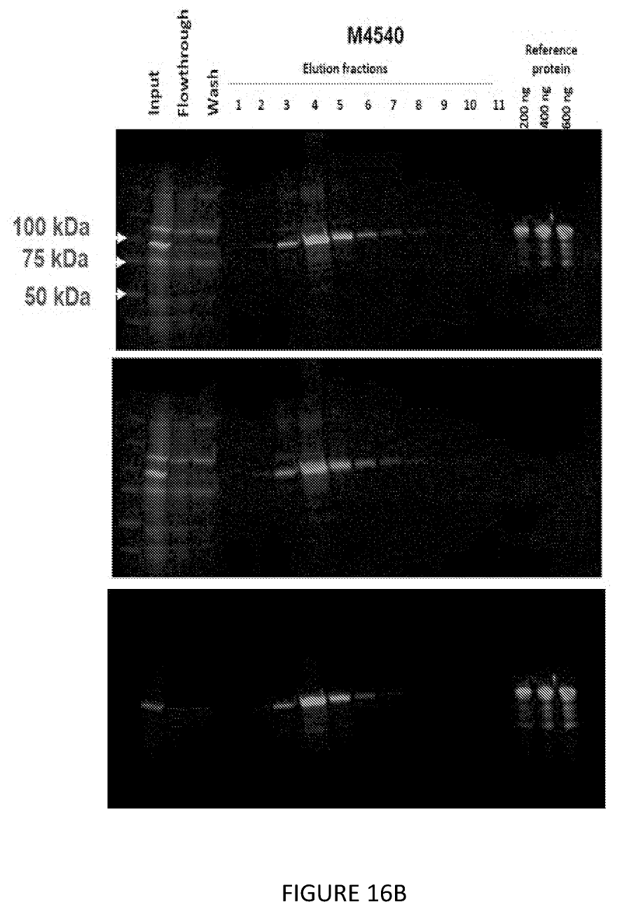

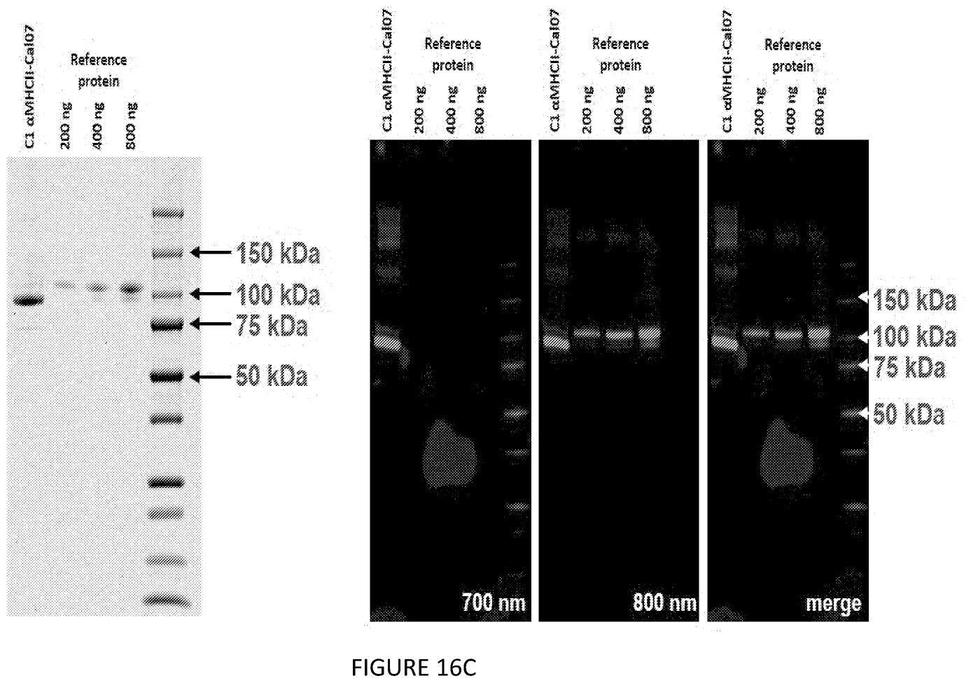

The C1 strain M4540 producing αMHCII-Cal07 recombinant protein was cultivated in 0.25 L bioreactor in a fed-batch process in a medium with yeast extract as an organic nitrogen source and glucose as a carbon source. The culture was performed at 38° C. for seven days. After ending the cultivation, the fermentation broth was stored at −80° C. For αMHCII-Cal07 purification by C-tag affinity chromatography, 50 ml of liquid culture was thawed on ice, and after thawing the sample was clarified by centrifugation 3×20 min at 20000×g at +4° C. followed by filtration through a 0.4504 filter. 33 ml of the clear supernatant was diluted with 1×PBS/0.5M NaCl (12 mM Na₂HPO₄·2H₂O, 3 mM NaH₂PO₄·H₂O, 650 mM NaCl pH 7.3) to final volume of 100 ml. The C-tag affinity purification was performed with 1 ml CaptureSelect C-tag XL column (Thermo Fisher) attached to ÄKTA Start protein purification system (Cytiva) and operated with a flow rate of 1 ml/min. Column was first equilibrated with 5 column volumes (CV) of 1×PBS/0.5M NaCl prior to loading the sample. After sample loading, the column was washed with 15CV of 1×PBS/0.5M NaCl and then eluted with one-step gradient of 10CV of 20 mM Tris-HCl, 2M MgCl₂, 1 mM EDTA pH7.5 with fraction volume of 1 ml. The quantity of the eluted αMHCII-Cal07 was quantified by integrating the UV trace of the elution peak with the Unicorn 1.0 software included in the ÄKTA Start system. The extinction coefficient of 1.7 was used in calculating αMHCII-Cal07 amount. After elution, the column was regenerated with 5CV of 0.1M glycine pH 2.3 and washed with 1×PBS till pH7.3 was reached. Elution fractions containing the protein were pooled for dialysis step to exchange the elution buffer to 1×PBS buffer. Pooled fractions were packed in a 12 ml dialysis cassette and the dialysis cassette was dialyzed in 1.5 L in 1×PBS for 1 h at +4° C. with stirring on a magnetic stirrer. 1×PBS was exchanged to fresh buffer after 1 h and dialysis was continued for 2 h under the same conditions. Finally, 1×PBS was exchanged and dialysis was continued overnight. Concentration of dialyzed αMHCII-Cal07 was determined with the Nanodrop spectrophotometer measuring absorbance at 280 nm and using extinction coefficient 1.7. Aliquots of RBD preparates were stored at −80° C. Affinity purification of αMHCII-Cal07 from M4540 fermentation supernatant is shown as an example in FIGS. 16A-16C. Primary agents CaptureSelect Biotin Anti-C-tag conjugate (ThermoFisher) and murine monoclonal antibody 29E3 raised against influenza HA antigen and secondary agents IRDye 680RD Streptavidin (Li-Cor) and IRDye 800CW goat anti-mouse IgG secondary antibody (Li-Cor) were used in Western detection.

### Example 11: Expression of SARS-CoV-2 RBD Variants in 14-Fold Protease Deficient C1 Strain

Three variants of Receptor Binding Domain (RBD) of SARS-CoV-2 spike protein were expressed in the protease deficient C1 strain DNL155. The three variants are: 1) RBD_B 0.1.1.7-UK having N501Y mutation, 2) RBD_B.1.351-SA having K417N, E484K and N501Y mutations and 3) RBD_1.1.28.1(P.1)-BR having K417T, E484K and N501Y mutations. The fragment of each variant was synthesized by GenScript (USA) and the optimized sequence of Wuhan RBD (in pMYT1142 Example 4) was used as the basis from which the mutated amino acids were replaced with the codon most frequent in C1. The synthetized fragment design was similar to the Wuhan RBD with C-tag (used in pMYT1142 Example 4) except that the Gly/Ser-linker between the RBD variant and the C-tag was three amino acids long where as in Wuhan RBD-C-tag the linker was five amino acids long. Variant RBDs were expressed as two gene copies in C1 and for the double copy expression in same genomic locus, two plasmid constructs (5′arm and 3′arm), both harbouring one gene copy, were made for each variant. In C1 cells, the recombination between the selection marker fragments within 5′ arm and 3′arm plasmids makes the marker gene functional and enables the transformants to grow under selection. For the 5′arm plasmids, synthesized fragments were amplified by PCR from the GenScript plasmids and cloned by Gibson Assembly (NEBuilder® HiFi DNA Assembly Cloning Kit, New England Biolabs) method into the PacI site of the C1 expression vector pMYT1055 under endogenous C1 bgl8 promoter and C1 chi1 terminator. The correct sequences of the constructs were confirmed by sequencing the fragments inserted into the plasmids. Plasmids of correct sequence were given the plasmid numbers pMYT1572 for RBD_B.1.1.7-UK, pMYT1574 for RBD_B.1.351-SA and pMYT1576 for RBD_1.1.28.1(P.1)-BR, respectively. For the 3′ arm plasmids, synthesized fragments in GenScript plasmids were cut out with MssI restriction enzyme and cloned by Gibson Assembly (NEBuilder® HiFi DNA Assembly Cloning Kit, New England Biolabs) method into the PacI site of the C1 expression vector pMYT1056 under endogenous C1 bgl8 promoter and C1 bgl8 terminator. Plasmids of correct sequence were given the plasmid numbers pMYT1573 for RBD_B.1.1.7-UK, pMYT1575 for RBD_B.1.351-SA and pMYT1577 for RBD_1.1.28.1(P.1)-BR, respectively.

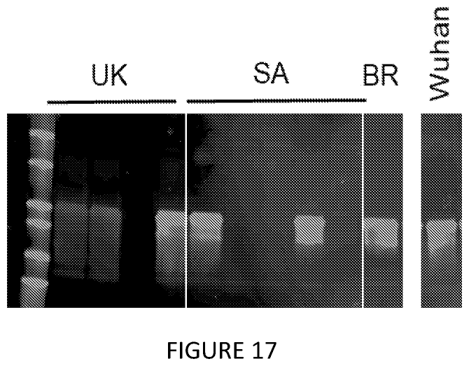

For double copy expression, both 5′arm and 3′arm plasmids were digested with MssI and plasmids harbouring the same variant gene were co-transformed to the DNL155 strain from which fourteen protease genes have been deleted. DNL155 was chosen as the host strain since production of Wuhan RBD was tested in several C1 protease deletion strains (Example 4) and the production was highest in DNL155 and DNL159 strains which are both 14-fold protease deletion strains with kex2 deletion. The transformation and screening of transformants by 24-well cultivation was performed as for Wuhan RBD (Example 4) except that culture supernatants were analysed by Western blotting with two primary detection agents simultaneously: SARS-CoV-2 (2019-nCoV) Spike RBD Antibody, Rabbit polyclonal antiserum (SinoBiologicals Cat. No. 40592-T62) and Capture Select Biotin Anti-C-tag conjugate (ThermoFisher). The secondary detection agents were Goat anti-rabbit IRDye 680RD (Li-Cor) and IRDye 800CW Stretavidin (Li-Cor). FIG. 17 shows an example of Western blotting result with at least one positive transformant for each RBD variant. Strong signals of the expected size with both primary antibodies are detected and production levels of the variant RBD-C-tag proteins appear to be equal to the M4169 control strain producing Wuhan RBD-C-tag.

RBD_B.1.1.7-UK amino acid sequence is set forth in SEQ ID NO: 53, and DNA sequence in SEQ ID NO: 54. The sequence includes a signal sequence, Gly/Ser linker and the C-tag.

RBD_B.1.351-SA amino acid sequence is set forth in SEQ ID NO: 55, and DNA sequence in SEQ ID NO: 56. The sequence includes a signal sequence, Gly/Ser linker and the C-tag.

RBD_1.1.28.1(P.1)-BR amino acid sequence is set forth in SEQ ID NO: 57, and DNA sequence in SEQ ID NO: 58. The sequence includes a signal sequence, Gly/Ser linker, and the C-tag.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A genetically modified ascomycetous filamentous fungus for producing an exogenous protein of interest, the genetically modified filamentous fungus comprises at least one cell having reduced expression and activity of at least one protease selected from KEX2 and ALP7, the at least one cell comprising an exogenous polynucleotide encoding the protein of interest.

2. (canceled)

3. (canceled)

4. The genetically modified ascomycetous filamentous fungus of claim 1, having reduced expression and/or activity of KEX2 and ALP7.

5. The genetically modified ascomycetous filamentous fungus of claim 1, wherein KEX2 comprises an amino acid sequence having at least 75%, or at least 80%, or at least 85%, or at least 90%, or at least 95%, or at least 99%, or 100% identity to the amino acid sequence of Thermothelomyces heterothallica KEX2, wherein the T. heterothallica KEX2, comprises the amino acids of SEQ ID NO:14.

6. (canceled)

7. The genetically modified ascomycetous filamentous fungus of claim 1, wherein ALP7 comprises an amino acid sequence having at least 75%, or at least 80%, or at least 85%, or at least 90%, or at least 95%, or at least 99%, or 100% identity to the amino acid sequence of Thermothelomyces heterothallica ALP7, wherein the T. heterothallica ALP7, comprises the amino acids of SEQ ID NO:13.

8. (canceled)

9. The genetically modified ascomycetous filamentous fungus of claim 1, having reduced expression and/or activity of at least one additional protease.

10. The genetically modified ascomycetous filamentous fungus of claim 9, wherein the additional protease is selected from the group consisting of ALP1, PEP4, ALP2, PRT1, SRP1, ALP3, PEP1, MTP2, PEP5, MTP4, PEP6, and ALP4.

11. (canceled)

12. The genetically modified ascomycetous filamentous fungus of claim 10, having reduced expression and/or activity of ALP1, PEP4, ALP2, PRT1, SRP1, ALP3, PEP1, MTP2, PEP5, MTP4, PEP6, ALP4 and KEX2.

13. The genetically modified ascomycetous filamentous fungus of claim 10, having reduced expression and/or activity of ALP1, PEP4, ALP2, PRT1, SRP1, ALP3, PEP1, MTP2, PEP5, MTP4, PEP6, ALP4 and ALP7.

14. The genetically modified ascomycetous filamentous fungus of claim 10, having reduced expression and/or activity of ALP1, PEP4, ALP2, PRT1, SRP1, ALP3, PEP1, MTP2, PEP5, MTP4, PEP6, ALP4, ALP7 and KEX2.

15. (canceled)

16. The genetically modified ascomycetous filamentous fungus of claim 1, wherein said genetically modified ascomycetous filamentous fungus produces the protein of interest in an increased amount compared to the amount produced by its non-genetically modified parent ascomycetous filamentous fungus strain cultured under similar conditions.

17. The genetically modified ascomycetous filamentous fungus of claim 1, wherein the protein produced by said genetically modified ascomycetous filamentous fungus has increased stability compared to said protein produced by the non-genetically modified parent ascomycetous filamentous fungus strain cultured under similar conditions.

18. The genetically modified ascomycetous filamentous fungus of claim 1, wherein the ascomycetous filamentous fungus is of a genus within Pezizomycotina.

19-21. (canceled)

22. The genetically modified ascomycetous filamentous fungus of claim 18, wherein the ascomycetous filamentous fungus is Thermothelomyces heterothallica C1.

23. (canceled)

24. The genetically modified ascomycetous filamentous fungus of claim 1, wherein the protein of interest is selected from the group consisting of an antigen, an antibody, an enzyme, a vaccine, and a structural protein.

25. (canceled)

26. The genetically modified ascomycetous filamentous fungus of claim 1, wherein the protein of interest is fused to a tag.

27. (canceled)

28. The genetically modified ascomycetous filamentous fungus of claim 24, wherein the protein of interest is a viral component.

29-33. (canceled)

34. A method for producing a fungus capable of producing a protein of interest, the method comprising transforming at least one cell of the fungus with at least one exogenous polynucleotide encoding the protein of interest; said at least one cell of the fungus having reduced expression and/or activity of at least one protease selected from KEX2 and/or ALP7.

35. The method of claim 34, said method further comprises engineering the fungus to have inhibited expression and/or protease activity of KEX2 or ALP7.

36-42. (canceled)

43. A method of producing at least one protein of interest, the method comprising culturing the genetically modified fungus of claim 1 in a suitable medium; and recovering the produced protein of interest.

44-47. (canceled)

48. A protein produced by a method according to claim 34.

49-50. (canceled)


SEQUENCE LISTING (58 sequences)

SEQ ID NO: 1 - protein, 392 aa, Thermothelomyces thermophilus
MHFSTALLAFLPAALAAPTAETLDKRAPILTARAGQVVPGKYIIKLRDGASDDVLEAAIGKLRSKADHVYRGKFRGFAGKLEDDVLDAIRLLPEVEYVEEEAIFTINAYTSQSNAPWGLARLSSKTAGSTTYTYDTSAGEGTCAYVIDTGIYTSHSDFGGRATFAANFVDSSNTDGNGHGTHVAGTIGGTTYGVAKKTKLYAVKVLGSDGSGTTSGVIAGINFVADDAPKRSCPKGVVANMSLGGSYSASINNAAAALVRSGVFLAVAAGNENQNAANSSPASEASACTVGATDRNDAKASYSNYGSVVDIQAPGSNILSTWIGSTSATNTISGTSMASPHIAGLGAYLLALEGSKTPAELCNYIKSTGNAAITGVPSGTTNRIAFNGNPSA

SEQ ID NO: 2 - protein, 397 aa, Thermothelomyces thermophilus
MKDAFLLTAAVLLGSAQGAVHKMKLQKIPLSEQLEAVPINTQLEHLGQKYMGLRPRESQADAIFKGMVADVKGNHPIPISNFMNAQYFSEITIGTPPQSFKVVLDTGSSNLWVPSVECGSIACYLHSKYDSSASSTYKKNGTSFEIRYGSGSLSGFVSQDTVSIGDITIQGQDFAEATSEPGLAFAFGRFDGILGLGYDRISVNGIVPPFYKMVEQKLIDEPVFAFYLADTNGQSEVVFGGVDHDKYKGKITTIPLRRKAYWEVDFDAISYGDDTAELENTGIILDTGTSLIALPSQLAEMLNAQIGAKKSYTGQYTIDCNKRDSLKDVTFNLAGYNFTLGPYDYVLEVQGSCISTFMGMDFPAPTGPLAILGDAFLRRYYSIYDLGADTVGLAEAK

SEQ ID NO: 3 - protein, 534 aa, Thermothelomyces thermophilus
MRGLVAFSLAACVSAAPSFKTETINGEHAPILSSSNAEVVPNSYIIKFKKHVDESSASAHHAWIQDIHTSREKVRQDLKKRGQVPLLDDVFHGLKHTYKIGQEFLGYSGHFDDETIEQVRRHPDVEYIERDSIVHTMRVTEETCDGELEKAAPWGLARISHRDTLGFSTFNKYLYAAEGGEGVDAYVIDTGTNIEHVDFEGRAKWGKTIPAGDADVDGNGHGTHCSGTIAGKKYGVAKKANVYAVKVLRSNGSGTMADVVAGVEWAAKSHLEQVQAAKDGKRKGFKGSVANMSLGGGKTRALDDTVNAAVSVGIHFAVAAGNDNADACNYSPAAAEKAVTVGASAIDDSRAYFSNYGKCTDIFAPGLSILSTWIGSKYATNTISGTSMASPHIAGLLAYYLSLQPATDSEYSVAPITPEKMKSNLLKIATQDALTDIPDETPNLLAWNGGGCNNYTAIVEAGGYKAKKKTTTDKVDIGASVSELEKLIEHDFEVISGKVVKGVSSFADKAEKFSEKIHELVDEELKEFLEDIAA

SEQ ID NO: 4 - protein, 307 aa, Thermothelomyces thermophilus
MKPTVLFTLLASGAYAAATPAIPGYSPRTRGMNPHHHAPLRLLHTFTPISTSGKSFRLLASSTESTKGGAILGLPDNDLSTVRTTIRIPAAKMPTAGPTANNTVGEYAASFWVGIDSATDACGAGGSLRAGVDIFWDGTLGGQQTPFAWYQGPGQADVVGFGGGFPVGEGDLVRLTLEAGPAGGEEIAVVAENFGRNVTRADEGAVPVRKVRKVLPAEAGGQKLCRGEAAWMVEDFPLQGRPEFPTALANFTSVTFNTGITLDDGTEKDLTGAEVLDIQLEAQGGRLTSCEVVDDRNVKCARVVGDN

SEQ ID NO: 5 - protein, 554 aa, Thermothelomyces thermophilus
MRIAASTVLLGAASAASFQQQAQHVLSDGFGKAQEAMKPLSDALADAAGRPIENFEEAFSGMTAEAKALWEEIKLLVPDSAFKNPSWFSKPKPHRRRDDWDHVVKGADVQKIWVQDANGESHRQVGGRIEDYNLRVKTVDPSKLGVDSVKQFSGYLDDEANDKHLFYWFFESRNDPKNDPVVLWLNGGPGCSSLTGLFLELGPSSIDKNLKVVNNEFSWNNNASVIFLDQPVNVGYSYSGSSVSNTIAAGKDVYALLTLFFHQFPEYAKQDFHIAGESYAGHYIPVFASEILSHKNRNINLKSILIGNGLTDGLTQYEYYRPMACGEGGYPAVLSESECRSMDNALPRCQSLIRNCYDSGSVWSCVPASIYCNNALIGPYQRTGQNVYDIRGKCEDSSNLCYSALGYISDYLNQQSVMDALGVEVSSYESCNFDINRNFLFQGDWMQPFHRLVPNILKEIPVLIYAGDADYICNWLGNRAWTEKLEWPGQKAFNQAKVHDLKLAGADEEYGKVKASGNFTFMQIYQAGHMVPMDQPENSLDFLNRWLSGEWFAK

SEQ ID NO: 6 - protein, 897 aa, Thermothelomyces thermophilus
MVRLDWAAVLLAATAVAKAVTPHTPSFVPGAYIVEYEEDQDSHAFVNKLGGKASLRKDLRFKLFKGASIQFKDTETADQMVAKVAEMPKVKAVYPVRRYPVPNHVVHSTGNVADEVLVKRQAAGNDTFSTHLMTQVNKFRDAGITGKGIKIAVIDTGIDYLHEALGGCFGPDCLVSYGTDLVGDDFNGSNTPKPDPDPIDNCQGHGTHVAGIIAAQTNNPFGIIGAATDVTLGAYRVFGCNGDTPNDVLIAAYNMAYEAGSDIITASIGGPSGWSEDPWAAVVTRIVENGVPCVVSAGNDGDAGIFYASTAANGKKVTAIASVDNIVTPALLSNASYTLNGTDDFFGFTAGDPGSWDDVNLPLWAVSFDTTDPANGCNPYPDSTPDLSGYIVLIRRGTCTFVEKASYAAAKGAKYVMFYNNVQQGTVTVSAAEAKGIEGVAMVTAQQGEAWVRALEAGSEVVLHMKDPLKAGKFLTTTPNTATGGFMSDYTSWGPTWEVEVKPQFGTPGGSILSTYPRALGSYAVLSGTSMACPLAAAIYALLINTRGTKDPKTLENLISSTARPNLFRLNGESLPLLAPVPQQGGGIVQAWDAAQATTLLSVSSLSFNDTDHFKPVQTFTITNTGKKAVTYSLSNVGAATAYTFADAKSIEPAPFPNELTADFASLTFVPKRLTIPAGKRQTVTVIAKPSEGVDAKRLPVYSGYIAINGSDSSALSLPYLGVVGSLHSAVVLDSNGARISLASDDTNKPLPANTSFVLPPAGFPNDTSYANSTDLPKLVVDLAMGSALLRADVVPLSGGAATATARLTRTVFGTRTIGQPYGLPARYNPRGTFEYAWDGRLDDGSYAPAGRYRFAVKALRIFGDAKRAREYDAAETVEFNIEYLPGPSAKFRRRLF

SEQ ID NO: 7 - protein, 566 aa, Thermothelomyces thermophilus
MKPSSAILLALAPGSSSKNVVEFSVSRGLPGNRTPLSFPPLTRRETYSERLINNIAGGGYYVQVQVGTPPQNLTMLLDTGSSDAWVLSHEADLCISPALQDFYGMPCTDTYDPSKSSSKKMVEEGGFKITYLDGGTASGDYITDHFTIGGVTVQSLQMACVTKAVRGTGILGLGFSISERASTKYPNIIDEMYSQGLIKSKAFSLYLNDRRADSGTLLFGGIDTDKFIGPLGVLPLHKPPGDRDYSSFEVNFTSVSLTYTNGSRHTIPTAILNHPAPAVLDSGTTLSYLPDELADPINTALDTFYDDRLQMTLIDCSHPLLRTDPDFHLAFTFTPTTSITVPLGDLVLDILPPTYPQSNSNNNNEVEDDDDDDDDDDDDDKVPPATERRWCVFGIQSTTRFAASSGQSEANFTLLGDTFLRSAYVVYDLSHYQIGLAQANLNSSSSSTNTNTIVELTADNHDDGASERGEGAGAGADAGTRTVIAGGLPSGLMGVEAQQTTFTPTATANGHPGYGGGPGGSTRPGSERNAAAGGFTAVRTGLLGELVGVAAVTALFILLGGALIAV

SEQ ID NO: 8 - protein, 874 aa, Thermothelomyces thermophilus
MAGGVNVQARELLPTNVIPRHYNITLEPDFKKLTFDGTVVIDLDVVEDSKSISLHTLELDIHDAKITSGGQTVSSSPTVSYNEDTQVSTFEFGNAVTKGSKAQLEIKFTGQLNDKMAGFYRSTYKNPDGSEGIMAVTQMEPTDARRSFPCFDEPSLKAEFTVTLVADKKLTCLSNMDVAYEKEVKSEQTGGIKKAVTFNKSPLMSTYLVAFVVGELNYIETNEFRVPVRVYAPPGQDIEHGRFSLNLAAKTLAFYEKVFGIEFPLPKMDQIAIPDFAQGAMENWGLVTYRVVDLLLDEKASGAATKERVAEVVQHELAHQWFGNLVTMDWWDGLWLNEGFATWASWYSCNIFYPEWKVWESYVVDNLQRALSLDSLRSSHPIEVPVKRADEINQIFDAISYSKGSCVLRMISTYLGEETFLEGVRRYLKKHAYGNTQTGDLWASLAEASGKKVEEVMQVWTKNIGFPVVTVTEKDDKTIHLKQNRFLRTGDTKPEEDQVIYPVFLGLRTKDGIDESQTLTKREDTFTVPSTDFFKLNANHTGLYRTAYSPERLKKLGDAAKEGLLSVEDRAGMIADAGALATSGYQRTSGVLSLLKGFNSEPEFVVWNEIIARVSSVQSAWIFEDQADRDALDAFLRDLASPKAHELGWQFSEKDGHILQQFKAMMFGTAGLSGDETIIKAAKDMFKKFMAGDRTAIHPNIRGSVFSMALKYGGTEEYDAVINFYRTSTNSDERNTALRCLGRAKSPELIKRTLDLLFSGEVKDQDIYMPASGLRSHPEGIEALFTWMTENWNELIKKLPPALSMLGTMVTIFTSSFTKKEQLERVEKFFEGKNTNGFDQSLAQSLDAIRSKISWIERDRADVTAWLKENGYRS

SEQ ID NO: 9 - protein, 454 aa, Thermothelomyces thermophilus
MKFAALALAASLVAAAPRVVKVDPSDIKPRRLGGTKFKLGQIHNDLFRQHGRGPRALAKAYEKYNIELPPNLLEVVQRILKDLGIEPHSKKIPGSKSSYGNGAPYTNETDDSGEVSAIPQLFDVEYLAPVQIGTPPQTLMLNFDTGSSDLWVFSSETPSRQQNGQKIYKIEESSTARRLSNHTWSIQYGDGSRSAGNVYLDTVSVGGVNVFNQAVESATFVSSSFVTDAASSGLLGLGFDSINTVKPTKQKTFISNALESLEMGLFTANLKKAEPGNYNFGFIDETEFVGPLSFIDVDSTDGFWQFDATGYSIQLPEPSGNITGTPFRAVAHTAIADTGTTLLLLPPGIAQAYYWQVQGARQAPEVGGWVMPCNASMPDLTLHIGTYKAVIPGELIPYAPVDTDDMDTATVCYGGIQSASGMPFAIYGDIFFKAQFTVFDVENLKLGFAPKPEL

SEQ ID NO: 10 - protein, 428 aa, Thermothelomyces thermophilus
MRVSFQSLLLLGALSAQASAYASLEYQQQTFPEDNAPPYRVPLLTLHRALVNVSSISDSEGEVGLLLKRLLKDLNYTVELQPVPPSEAGQGPDDRPTRYNVLAWPGRNASRALDKRTIITSHIDVVPPYIPYAIDNETVPPSEVVDFAALPPTTLISGRGSVDAKASVAAQITATNALLSEGAISPDSVVLLYVVGEENSGSGMKHFSDSLSNSSAYPVRPQFRAAIFGEPTENKLACGHKGVTGGTVSAVGKAGHSGYPWLGKSAIHVLIRALDRLLEEDLGSSERYGNTTVNVGLIEGGVAANVIAPAASARVSARVAVGNQTTGGQIVAERIKKLIKDVDSEALQVNITSGVGPVECECEVDGFETVVANYGTDIPNLKGNHVKYLYGPGSILVAHGDNEGLQIKDLEDSVEGYKRLIKHAVGSS

SEQ ID NO: 11 - protein, 444 aa, Thermothelomyces thermophilus
MEIEIGTPPQKVMLIVDTGSPNTWVNPQCETSNTPSDCAKYPQFDYTESSSINITDYVDVLRYGSGSATVQYVYETVSIGSATLKDQIIGIALESEDIPLGILGLSPPVRGVNQYPYILDTMVDQGLIKSRAFSLDLRGVDNPTGAVIFGGIDTGKYIGTLAKLPIIAPSSAPGGADRYYITMTGVGLTLPDGTMVRSEELDVPVFLDSGSTLSRLPTVIHQALAASFTEAMLDQESGLFILPCEYTDMAGSIDFYFAGKTIRVPLREFIWRSGDYCILGVAPEDDEPILGDTFLRAAYVVYDQDNRNVHLAQAADCGTNLVAIGSGEDAVPSSTGRCTELPTPTGDPTRTRAGSSNLDMTATRPPANTFTGRLPTGIAGGPGPARDGSTTTVTGGGLQPMLPTGSPKGSEGTEQNAAGRGVDSGLGAAVAAVLGVVSLLVLML

SEQ ID NO: 12 - protein, 621 aa, Thermothelomyces thermophilus
MLRNIFLTAALAAFGQCGSTVFESVPAKPRGWTRLGDASADQPLRLRIALQQPNEDLFERTLYEVSDPSHARYGQHLSRDELSALLAPRAESTAAVLNWLRDAGIPSDKIEEDGEWINLRVTVREASELLDADFGVWAYEGTNVKRVRALQYSVPEEIAPHIRMVAPVVRFGQIRPERSQVFEVVETAPSQVKVAAAIPPQDLDVKACNTSITPECLRALYKVGSYQAEPSKKSLFGVAGYLEQWAKYDQLELFASTYAPYAADANFTSVGVNGGENNQGPSDQGDIEANLDIQYAVALSYKTPITYYITGGRGPLVPDLDQPDPNDVSNEPYLEFFSYLLKLPDSELPQTLTTSYGEDEQSVPRPYAEKVCQMIGQLGARGVSVIFSSGDTGVGSACQTNDGKNTTRFLPIFPGACPYVTSIGATRYVEPEQAAAFSSGGFSDIFKRPAYQEAAVSTYLHKHLGSRWKGLYNPQGRGFPDVSAQGVAYHVFSQDKDIKVSGTSASAPLFAALVSLLNNARLAQGRPPLGFLNPWLYSEKVQKAGALTDIVHGGSSGCTGKDMYSGLPTPYVPYASWNATPGWDPVTGLGTPVFDKLLELSSPGKKLPHIGGGHGHGAGGH

SEQ ID NO: 13 - protein, 420 aa, Thermothelomyces thermophilus
MAGRLLLCLTAALSALGVSAAPAPDASGRPFIGVPVSNPGIANAIPNRYIVVYNNTFNDEDIDLHQSNVIKTIAKRNIAKRSLTGKLLSTTVNTYKINNWRAMALEADDATINEIFAAKEVSYIEQDAVISLNVRQMQSQATTGLARISHAQPGARTYIFDSSAGEGITAYVVDTGIRVTHEEFEGRATFAANFIDDVDTDEQGHGSHVAGTIGGKTFGVAKKVNLVAVKVLGADGSGSNSGVIAGMQFVASNATAMGLKGRAVMNMSLGGPASRAVNSAINQVEAAGVVPVVAAGNESQDTANTSPGSAEAAITVGAIDQTNDRMASFSNFGELVDIFAPGVNVQSVGIRSDTSTNTLSGTSMASPHVAGLAAYIMSLENITGVQAVSDRLKELAQATGARARGVPRGTTTLIANNGFA

SEQ ID NO: 14 - protein, 892 aa, Thermothelomyces thermophilus
MKIWSGAALLGLAALATASHILPRDWEANDYYVLHLDADTSPQEVARSLGLSHEGPLGELRDHHVFVAKRAEHDVVKRELARRRKKRSLGLGGRDVLDGVLFSQKQRLRKPWEKRVVPRLFGPLPRRSVDEPVESLVQRQTEVARKLDIKDPIFHEQWHLFNTVQAGHDVNVTDVWLQGVTGKNATVAIVDDGLDMYSDDLRDNYYALGSYDFNDKADEPRPRLANDNHGTRCAGEVAAGRNNACGVGVAYDSNIAGLRILSKLISDADEAVALNYDFQHNQIYSCSWGPPDDGKSMDAPGILIRRAMLNAVQNGRGGLGSIYVFASGNGAHNEDNCNFDGYTNSIYSITVGALDRKGQHPYYSESCSAGLVVTYSSGSGDAIHTTDVGQNTCTSSHGGTSAAAPLAAGIFALVLQVRPDLSWRDMQYLAMDTAVPVNVDTGDYQDTTIGKKFSHTYGYGKLDSYAIVEAAKKWKKVKPQAWFYSPWIHVNQPIPQGDKGVVVEFEVTKEMLEEANLDRLEHVTVTMNVEHGRRGDLSVDLISPNKIVSHLSVTRKNDDSDKGYNDWTFMSVAHWGESGVGTWTIVVKDTEINQYTGKFIDWHLKLWGETRDASKAQLLPMPTEEDDDDHDVIATTTATAATTTVSKPEATGSVPADATDQPNRPVNSKPTDTSPAETSSSSSSSSSSAETDKTNTWLPSFLPTFGVSAATQAWIYGSLVLIVLFCAGLGIYLYLARRKRLRNKTRTDYEFELLDDDDDDDEEAAALTRGGGGGEKGVVGGGGGGGGKRGRRTRGGELYDAFAGESDEDSDDNDFAGGGGGGGGGGYRDRSDSRSRSRSDGSGSPIGISEKLPGRRDSLSGEEEHHVVGDDDDDDEDGTGNDQARPLQGGAR

SEQ ID NO: 15 - protein, 387 aa, Thermothelomyces thermophilus
MQLLSLAALLPLALAAPVIKPQGLQLIPGDYIVKLKDGASESTLQDTIRHLQAGEAKHVYRARRFKGFAAKLSPQVVDTLSKLPEVEYIEQDAVVTIQALVTQEDVPWGLARISHHELGPTSYVYDDSAGEGTCAYVIDTGIYVAHSQFEGRATWLANFIDSSDSDGAGHGTHVSGTIGGVTYGVAKKTKLFAVKVLNASGSGTVSSVLAGLEFVASDAPARVASGECANGAVANLSLGGGRSTAINAAAAAAVDAGVFVAVAAGNSNTDAQSTSPASEPSVCTVGATDDSDARAYFSNYGSVVDVFAPGVDVLSSWIGGVDATNTISGTSMATPHIAGLGAYLLALLGPRSPEELCEYIKQTATIGTITSLPSGTINAIAYNGATA

SEQ ID NO: 16 - protein, 1468 aa, Thermothelomyces thermophilus
MVASSWFTAPLVAVALLLSLDGAVAKKPTFRPPSLPTYDDDAACPERCSVSGPSTGNWSVYPNFEPIRKCTQTMFYDFSLYDSVDDPTVNHRIHACSSFGPDFSIIPGSITKTAYASPAPAKIRFELGWWNRGYGLAAPGLRSLVKQLRAYIDHGHGDGAADRPFIIYGQSGQATIGLYIGQGLLSQGLSKSALKILQDNLANSDVSAPSLAIQLCGQGYGSSHIFGAMVTSNGTFAPIQEAIRTWANATCLSFAGSKEFPGEVMFTTPLLLANGTANSTVRARSLRPYAAECRTVQVEAGDSCGTLAKKCGISGADFTNYNPGASFCSTLKPKQHVCCSSGTLPDFRPVTNPDGSCYSYKVKSNDNCADLAAEYGLTVDEIESFNKNTWGWGGCKVLFLDTIMCLSKGAPPFPAPISNAICGPQKLGTIPPTDGSNIADLNPCPINACCNIWGQCGISKDFCIDTNTGPPGTAAPGTYGCISNCGLDIVKGKGTGSIKIAYFEGFGLERECLFRDASQIDRSKYTHVHFAFGTLTPTYEVNVGDILSSYQFTQFKLISGPKKILSFGGWDFSTSKATYSIFRNGVKAENRLTMAKSIANFIKEHDLDGVDIDWEYPGAPDIPDIPAGEEDEGTNYLAFLVVLKNLLPGKSISIAAPSSYWYLKQFPIKAISRIVDYIVFMSYDIHGQWDAHNMWSQDGCVTGNCLRSHVNLTETRLALVMITKAGVPGEKVIVGVTSYGRSFDMAQPGCWSPDCQFTGDRLNSNAKPGRCTGTAGYISNAEIDEILAGGGSSGGSSQARAGRVVASFVDTSSNTDVLVYDNNQWVGYMSEKTKKTRTTLYTGWGLGGTTDWASDLQQYHDVPGPAKDWTEFKQLIRAGEDPKSDHSREGDWTKFDCTNPYLVDKTFYTPTQRWKNLDTDAAWRDVVRIWKETDKPRNIMFTASVSTTLYISADVDCRNLEDCNTTEECSAGLNGPYSGPAAQFIWNSMVKIHAMYHNYVLMLERATSLVSMALDDMQKTFAPVPVEEDKAWLYLLIDLITLGTLTVAGPLYNRQLGMYVYFSDKSVDDIKDTTMTLIGQSTTIAKDVLSTKQEAWTENLQASFNNMLSRVIEGWQNATSLAVNKIFSGSETSLNILWDVMSDGKLIEGMPPPGSGPPPDPGNIHNELQANVKKSIYAFAIPNLWRVSQTFAFILDSGFGCDVEKPLQDYLEDETMEATGACVDGKRYYLVAPIGESRTCDWVNGMWDCTLSNKFSAPPGLDRLGADFGYLTKEDFIKGSIRTWLKNGKRNAGGGMPDVTDIDTINSLIDLDFTTPGFIHLPVCSPERAYQTWDTSSSGYGANYPCDPPPGINNCGDSTFEDQTSAASPKVEDCLQIIKNIQDDGKTEWTIQVLGKNQREIAKFGECRFGVEATEQTGNADFKVGGQDVIDIINDAVEKFGGSGRVGAKGDMSCNGNIKGQAVKWGIY

SEQ ID NO: 17 - protein, 561 aa, Thermothelomyces thermophilus
MLRGTIAVGVACLAQLVAGLDGPLFRTSLTLRDFREQLERRQARDGAALEARSSDLQDLYPAHTLQVPVDHFHNDSLYEPHSSETFPLRYWFDASHYKKGGPIIVLQSGETDGVGRLPFLQKGIVAQLARATNGLGVILEHRYYGESIPTPDFSTEKLRFLTTDQALADMAYFARHVVFKGLEHLDLTSAKNPYIAYGGSYAGAFVAFLRKLYPDVYWGAISSSGVPEAIYDYWQYYEAARIYAPHDCVVATQKLTHIVDNILLDKADTDYVRRLKTGFGLGGVTRNDDFANAISWGIGGLQGLNWDPALNDTGFGEYCNNLTATKPLYPTSPALEQEARELVKAGGYGKEADTLTTQLLNYMGYVNATTVQTCHKDSQDECFTNYNSTFYQQDDKTQDWRLWPYQYCFEWGYLQTGSGVPANQLPLISRLIDLNFTSVVCREAFNITTPSQVERINKLGGVNISYPRLAFVDGERDPWRYASPHRIGLPERKNTISEPFILIKDGVHHWDENGLFPNETRPGLPPKPVADAQRAEVKFVKAWLKEWKEKEKCRGRKFCWP

SEQ ID NO: 18 - protein, 640 aa, Thermothelomyces thermophilus
MTMKGSTLLALALGFGAHAQFPPKREGITVIESKFYKNVSISFKEPGICETTPGVKSYSGYVHLPPNLIEGADQDYPINTFFWFFEARKDPANAPLAIWLNGGPGGSSMMGLLEENGPCFVGPDSKTTYLNRWSWNNEANMLYIDQPVQTGFSYDVLTNVTVQLDVDDPSEPIITPTNFTDGHIPRTNNTFRIGTVGSQKASQVTNSTELSAHAMWHFLQTWLFEFPHYRSDDGRISLWAESYGGTYGPAFFRFFQQQNERIADGQLEGRYLHLDTLGIINGAVDWPILAESLIDYPYNNSYGIQFYNDTFHAALKHNWTRPSGWREQMQACTESLASSSSSSSPPAAGCEAVRSVLDDVLAAAFPRQSGRAPFDLAHPRADPFPPPHPHGFLARADVQAALGVPVNHTAVSLPVNRAFDATFDPLRGGQLDALAGLLDRRAGGGVKVHLVYGDRDPSCNWAGGEKVSLAVPWSRRDVFAAAGYAPLVVVSGKGGGDGGNTGGGNTGGGEEEVVVVRGLTRQVGRFSFTRVFQAGHEVPSYQPQAGYEIFRRAMAGLDLPTGRVRAGDDFVTAGLRDAWAVKNAAPDMVEPRCYVLKPESCEPEVWKTVVDGTAIVKDWFVVGSTGGEGRGVEGGIDGDE

SEQ ID NO: 19 - protein, 571 aa, Thermothelomyces thermophilus
MLWTTLLSALLLTGTAEAAGRSIAHAGKRHVEHAAKRAKPIMPAGPYHPVIEREEKAPKFLTPKTEKFAVDGKGIPDVDFDVGESYAGLLPLSSDPNDDKNLFFWFFPSTNPAAEKEILIWLNGGPGCSSFEGLLQENGPFLWQYGTYKPVQNPWSWHTLTNIVYVEQPVGTGFTTGTPTITNEEELAAEFMGFWKNFVDTFGLHGYKVYIAGESYAGYYCPYIAAAFLDEEDKTYYDMSGMTIYNPSLAPDEIQEPIPVVAFTEYWSGLFPFNDTFRADIKRREKECGYADFLAEYLVYPPKGPLPSRLPGTHRDGTTREECWNIYWDIFDAISVLNPCFDIYQVATTCPLLWDVLGFPGSMPYLPEGTKVYFDREDVKRAIHAPVNATWEECSSRDVFVNGTDHSVPSTVRALPRVIDGTKNVIIGHSALDMILLANGTLLALQNMTWGGKRGFQSRPDQPFYVPLNNITTLSTLAAAGVFGSLVSERGLTYVGVDLAGHMVPQYAPSAAYRHVEYMLGRVDCMNCTKPFTTDPFTPQSKGKLGKGTAPQGWSNASGHGKGNGPRRIRA

SEQ ID NO: 20 - DNA, 8017 bp, Thermothelomyces thermophilus
agggtaggtgggatgggcggggtgtagggtaggtcggtgtagggtaggtcggctgggcggggtgtagggtaggtcggttgggcggggtgtagggtaggtcggttgggatgggtgtagggtaggtcggccgggtgtagggtaggtcggctgggcggggtgtagggtaggtcggtgtagggtaggtgggatggggcgctatgtgcggccgcgagctcgcgagcccatttttagcgaaggccatacaaacgagttttgcggaacccgggattccacccccgaagccgccggcgcgtgcgccccgctgcgcatcggtcggtgggtatatgagaagggggcgggcaagccggaagccagaggcaactgctactgttagctgccgctggcctccgcggcccagggcgcggcacggctgcgttgaagtctcccagtctcccacccgttggctgcgcggatccgcccgtcttggtggttgcgagctcgcgagcccatttttagcgaaggccatacaaacgagttttgcggggcccgggattccaccccggaacccgccggcgcgtgcgccccgctgcgcatcggtcggtgggtatgtgagggaggaagaagaaaaaaaaaaaaagctcctgcgggggggctgtcgggcacgcctactttcgggcgacccggcacctctccgcggcagccttcgcaggccgctgttggtcccatttcatacgtcgccgccttcgcgtggtgccctacggtctgccggggtaccgacgattgcggcgagcaccgcctcagcaccgctgctgccaccggcgcgacctcgcccgggggtgcgcgcggcatctgggaagactctgcaggcgtaagggaataccccatgtgcgccgaggggtgggctatgtgggtgcttggcggttcgccagacctttctaaagccaccgggggtacctaccggttggggacgcctacagggctgaacctcccggtcgggcctcctcttggggcgcttaggcggcgacttcggggcgcgatcgctccccgctctcgcccgccgacggcgctctggggaattcaggaggggaaagcagatgtgacccgcggctcgaccggcgcattgccggacgagctgcgcggccacgcgggcccccgcgcccgccgacccagtaacttagtgaactcttccgccctgaaacacgggcggttggccctaaccggctcacgatagttacctggttgattctgccagtagtcatatgcttgtctcaaagattaagccatgcatgtctaagtataagcaattatacagcgaaactgcgaatggctcattaaatcagttatcgtttatttgatagtaccttactacatggataaccgtggtaattctagagctaatacatgctaaaaatcccgacttcggaagggatgtatttattagattaaaaaccaatgccctccggggctctctggtgattcatgataacttctcgaatcgcacggccttgcgccggcgatggttcattcaaatttctgccctatcaactttcgacggctgggtcttggccagccgtggtgacaacgggtaacggagggttagggctcgaccccggagaaggagcctgagaaacggctactacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgaggttaaaaagctcgtagttgaaccttgggcctagccggccggtccgcctcaccgcgtgcactggctcggctgggtctttccttctggagaaccgcatgcccttcactgggtgtgccggggaaccaggacttttactctgaacaaattagatcgcttaaagaaggcctatgctcgaatacattagcatggaataatagaataggacgtgtggttctattttgttggtttctaggaccgccgtaatgattaatagggacagtcgggggcatcagtattcaattgtcagaggtgaaattcttggatttattgaagactaactactgcgaaagcatttgccaaggatgttttcattaatcaggaacgaaagttaggggatcgaagacgatcagataccgtcgtagtcttaaccataaactatgccgattagggatcggacggcgttattttttgacccgttcggcaccttacgataaatcaaaatgtttgggctcctgggggagtatggtcgcaaggctgaaacttaaagaaattgacggaagggcaccaccaggggtggagcctgcggcttaatttgactcaacacggggaaactcaccaggtccagacacgatgaggattgacagattgagagctctttcttgatttcgtgggtggtggtgcatggccgttcttagttggtggagtgatttgtctgcttaattgcgataacgaacgagaccttaacctgctaaatagcccgtattgctttggcagtacgccggcttcttagagggactatcggctcaagccgatggaagtttgaggcaataacaggtctgtgatgcccttagatgttctgggccgcacgcgcgctacactgacagagccagcgagtactcccttggccggaaggcccgggtaatcttgttaaactctgtcgtgctggggatagagcattgcaattattgctcttcaacgaggaatccctagtaagcgcaagtcatcagcttgcgttgattacgtccctgccctttgtacacaccgcccgtcgctactaccgattgaatggctcagtgaggctttcggactggcccagagaggtcggcaacgaccactcagggccggaaagttatccaaactcggtcatttagaggaagtaaaagtcgtaacaaggtctccgttggtgaaccagcggagggatcattacagagctgcaaaactccctaaaccatcgtgaacgctacctagaccgttgcttcggcgggcggcgccctcgcgcgccccccctggggcccgcaccgcgggcgcccgccggaggtacaccaaactcttgatatgttatggccactctgagtctcctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcatcctggcgggcatgcctgttcgagcgtcatttcaacccatcaagcccacggcttgtgttggggacctgcggctgcccgcaggccctgaaaaccagtggcgggctcgctagtcacaccgggcgtagtagcatacgacctcgctcagggcgtgctgcgggttccagccgtaaaacgaccttcacaacccaaggttgacctcggatcaggtaggaggacccgctgaacttaagcatatcaataagcggaggaaaagaaaccaacagggattgccctagtaacggcgagtgaagcggcaacagctcaaatttgaaatctggcttcggcccgagttgtaatttgcagaggaagctttaggcgcggcaccttctgagtcccctggaacggggcgccatagagggtgagagccccgtatagttggatgcctagcctgtgtaaagctccttcgacgagtcgagtagtttgggaatgctgctcaaaatgggaggtaaatttcttctaaagctaaataccggccagagaccgatagcgcacaagtagagtgatcgaaagatgaaaagcactttgaaaagagggttaaatagcacgtgaaattgttgaaagggaagcgcttgtgaccagacttgcgccgggctgatcatccggtgttctcaccggtgcactctgcccggctcaggccagcatcggttctcgcggggggataaaggcccggggaatgtagctcctccgggagtgttatagccccgggtgtaataccctcgcggggaccgaggttcgcgcatctgcaaggatgctggcgtaatggtcatcagcgacccgtcttgaaacacggaccaaggagtcaaggttttgcgcgagtgtttgggtgtaaaacccgcacgcgtaatgaaagtgaacgtaggtgagagcttcggcgcatcatcgaccgatcctgatgttttcggatggatttgagtaggagcgttaagccttggacccgaaagatggtgaactatgcttggatagggtgaagccagaggaaactctggtggaggctcgcagcggttctgacgtgcaaatcgatcgtcaaatctgagcatgggggcgaaagactaatcgaaccatctagtagctggttaccgccgaagtttccctcaggatagcagtgttgtcttcagttttatgaggtaaagcgaatgattagggactcgggggcgctttttagccttcatccattctcaaactttaaatatgtaagaagcccttgttacttagttgaacgtgggccttcgaatgtatcaacactagtgggccatttttggtaagcagaactggcgatgcgggatgaaccgaacgcggggttaaggtgccggagtggacgctcatcagacaccacaaaaggcgttagtacatcttgacagcaggacggtggccatggaagtcggaatccgctaaggactgtgtaacaactcacctgccgaatgtactagccctgaaaatggatggcgctcaagcgtcccacccataccccgccctcagggtagaaacgacgccctgaggagtaggcggccgtggaggtcagtgacgaagcctagggcgtgagcccgggtcgaacggcctctagtgcagatcttggtggtagtagcaaatacttcaatgagaacttgaaggaccgaagtggggaaaggttccatgtgaacagcggttggacatgggttagtcgatcctaagccatagggaagttccgtttcaaaggggcactcgtgccccgtgtggcgaaagggaagccggttaacattccggcacctggatgtgggttttgcgcggtaacgcaactgaacgcggagacgacggcgggggccccgggcagagttctcttttcttcttaacggtctatcaccctggaaacagtttgtctggagatagggtttaacggccggaagagcccgacacttctgtcgggtccggtgcgctctcgacgtcccttgaaaatccgcgggagggaataattctcacgccaggtcgtactcataaccgcagcaggtccccaaggtgaacagcctctggttgatagaacaatgtagataagggaagtcggcaaaatagatccgtaacttcgggataaggattggctctaagggttgggcacgttgggctttgggcggacgccctgggagcaggtcgcctctagccgggcaaccggcggggggcttccagcatccgggtgcagatgcccttagcaggcttcggccgtccggcgcgcggttaacaaccaacttagaactggtacggacagggggaatctgactgtctaattaaaacatagcattgcgatggccagaaagtggtgttgacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacataggaggtgtagaataggtgggagcttcggcgccggtgaaataccactactcctattgtttttttacttattcaatgaagcggggctggattttcgtccaacttctggttttaaggtccttcgcgggccgacccgggttgaagacattgtcaggtggggagtttggctggggcggcacatctgttaaaccataacgcaggtgtcctaaggggggctcatggagaacagaaatctccagtagaacaaaagggtaaaagtccccttgattttgattttcagtgtgaatacaaaccatgaaagtgtggcctatcgatcctttagtccctcgaaatttgaggctagaggtgccagaaaagttaccacagggataactggcttgtggcggccaagcgttcatagcgacgtcgctttttgatccttcgatgtcggctcttcctatcataccgaagcagaattcggtaagcgttggattgttcacccactaatagggaacgtgagctgggtttagaccgtcgtgagacaggttagttttaccctactgatgaactcatcgcaatggtaattcagcttagtacgagaggaaccgctgattcagataattggtttttgcggttgtccgaccgggcagtgccgcgaagctaccatctgctggataatggctgaacgcctctaagtcagaatccatgccagaacgcgatgatactacccgcacgttgtagacgtataagaataggctccggcctcgtatcctagcaggcgattcctccgccggcctcgaagttggccggcggtaattcgcgtattgcaatttcgacacgcgcgggatcaaatcctttgcagacgacttagatgtgcgaaagggtcctgtaagcagtagagtagccttgttgttacgatctgctgagggtaagccctccttcgcctagatttcccagcgagagcccgccggcggaacagccgggcgagccttacgggggaagccttaaggggattgagaagtggtgccgtgcgttcgcgcgcccctaggtcctttagccggccgcaggtgtagggtaggtcggttgggaggatggggtgtagggtaggtcggtgtagggtaggttggttgggaggatggggtgtagggtaggtcggccgggtgtagggtaggtcggtgtagggtaggtgggatggggcgctatatgcggccgcgagctcgcgagcctatttttagtgaaggctatataaataagctttacgttaccgggccttgctaccctcgagtggcgtgggccgtgctgcctactgggcattgctcgccgggctgtataagggaggggtcggggtcgcggtctagggtaggtcgggtgggatggggtgtagggtaggagaagcgctctagtcgtgtgtctttttctctaggtctattattagtactggctgtagggcgacgtgccctgccttgttataatattatattgtatgtttaggcctatactagcttgtaatctatttgtatctggcttattaggtacggcttcctttgtatataactagagaggctctggtatgcttcttagtatagcggtataggattcataatcatagtaatgataatcataatagtaataataataataatagtaatgataataataataatctatttatatcttatttaaaatgcttgtacggctgcctgctcttaaggagtagctagatatgagatggtagggtagctagctaacctaggctagacgttctcgtcccttagctatataagtgctatatattatagttagttatctaacctaccttcttacttgagcagaagaggtagggttctagtatagctagtagggcttctaggcctaagggcctgttattcgagttattataggttagtatttaatatagttatagggataggcctcgattacgggtataggataggtaggataggtatagggtaggtcggttaggaggatagggtgtaaggtaggtcggccgggtatagggtaggtagtaggttaggcggggtgtagggtaggtcggtgtagggtaggtgggatgggcggggtgtagggtaggtcggttgggaggatggggtgtagggtaggtcggtgtagggtaggtcgg

SEQ ID NO: 21 - DNA, 8334 bp, Artidicial sequence
gtttaaacgaaaggatctctcgcccaggtggacaacccgcataatggaggcgccgtggtggattttgccatgcaggagccaaccgccagcggccatgaacccgcccagcacgatcactctaccaagtaacgttaaagaggcgatccctcatgcgccggagtaactaacggagtaccttctcattgatcttactgacttgttagtccgcgctgacggccaacagttcaaccagcccggtgatcagcagctcgagggcttgcactggccatccgccgggcgcttggctgagcgagcatccgcgggtcagccacagccgccggtctgatgtggccgtggcatgacctcagcttgtttatgggttattagatctggcttagatccggcttatttagatatctatctagtcgaggcagggggttcgagagaccgtttggtttggttcatccgacgtttgcctctctgagctcggtgagtgagagcttcggttgcgacggccaagcatcgcggggccgcactcgcaggctgcttcgcaccacggaactcatccgagctacggagtccctccgtacgctactcgagtacgtccgtcctctgtaggcatctcgagccgtcgccagcatttgagaccaatggaagggggtttcctcccgtgcaactttggttgaaaccttcaaagcgtcccccgactggggcaaccgtttgacttggatccacacttcaggggtagagaggctcctctcgacaaccccctgtatttctgtaaccccccttgaacgccgtacatggcaccacaacaccaaacaagccatgctgcttggaatcttcgctccgaaaagagccccccccgggccctccaacttcatgcgctccctcctcagttaaatagcggccgctctgctccatggagtattgctctcttcattaggctgttgtgatttgcttgcttgttttcctttccttttccgcacacccattccctcttttcactgggtcgaggctctctccaccgataaccttcgatccgcggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 22 - DNA, 8273 bp, Artificial sequence
  DNA sequence
ggcgcgccgtttaaacgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccttcgcaccacggaactcatccgagctacggagtccctccgtacgctactcgagtacgtccgtcctctgtaggcatctcgagccgtcgccagcatttgagaccaatggaagggggtttcctcccgtgcaactttggttgaaaccttcaaagcgtcccccgactggggcaaccgtttgacttggatccacacttcaggggtagagaggctcctctcgacaaccccctgtatttctgtaaccccccttgaacgccgtacatggcaccacaacaccaaacaagccatgctgcttggaatcttcgctccgaaaagagccccccccgggccctccaacttcatgcgctccctcctcagttaaatagcggccgctctgctccatggagtattgctctcttcattaggctgttgtgatttgcttgcttgttttcctttccttttccgcacacccattccctcttttcactgggtcgaggctctctccaccgataaccttcgatccgcaggcgcgccccccatgtttcccaaagggtcctgtttgtttgttttttcttcctcttctttaagggctcgggtgacagctggagagccgagcaagcagcacataaaatggcttgcgaattcaggatacattgattatgtcatggacccaaggaaacaccctcttcctgcgccgtccactgcaccaacttctcctcaaacacgatcaaccacgtaggaacagcagacgaaaacgtcacctggccgcgattgacatacccaaagtgagacgtggaggagacgggggttgcgtgcttcggccggatgaggttttcagaccgactacggtactggatcttagcagcacaaagatcacctacccagagtaagtagttggacaagcgctttcactcggaactcagagccaacggaaatcggatgaggcatcaagatttttcgaggggcaactactccgtcggacaaccgagtcctgtgtgcaagcgccgcatttcgtcattgtagatgttggagacatgtttgcagtccgccctaagcaggccattccgtcggagaagagggaaagacccggccgagggcgtgcccgggttccaggctacttgccacgagggtttcatatcagcacatcttcggcacaaccacagccattgagcccgattgccccgagaggggaaagggcggctgaattgcaatttgacatccgtgcttgttgttacccttgtttagcaaagccagtgggggtcatcaataccacctccaaggcgcgcatatcacggcaacacctggcccgataaaacagaagccaaacacgtgtgtattatgttggtattagatgttcgcttctcccaaccggagctgatgcccgcgccagatcccgcgcccaacagttcactcgtaatcgttgtatacatgaccgcctgtatcgaagggtatgtgtcattagcaaggtatatagaaacgtgaaccgaaaatgctcatctcgccggtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 23 - DNA, 8375 bp, Artificial Sequence
  DNA sequence
gtttaaacgtttaaacggtggacacaaacagttgccaattagatctcctgaacttaataaagcgccattctgttgaaagacgagatcgccgagcgtaccaagtcttgttcgggctctgaacaaaactggaaccaccactccaaaacggtagttgatgcgaatacctatatcctccagatcgagtctgcagtgcagcgtggatatgggtatcggcacttgcgtgcacagtagtgtagctccgtagtacggagtgctgcaggatatcaacttctaagccacagccgttccgagcggaacatttcttcattggctccctcgcgcatggggcaggtcgtgcagtgttcgtggctatttgcggatcatacagacaccttttggggtccgcttcgggaaactgccgcccccctacactacggagtagtgcgacgccgcattcccgttcggcgcttggcagccaattctaccactcggggccgtctgcaaggccacctctgcattttcaagtcgaatagtcaatagtcacaaaaactggtcaaactggccaaactggtcaacccggttgccaccttttggaaagagcaccgcttctttttcgttcctcggcctgagccgtcgaatgcgaacgtcaaaaggcgaactagaaattctgaaacatagtacggattactccgtacccggttgttttgcaccgggattttgcttcaatcgccaccgagttccacccactttcgccaaggtacggattacagtaatccgtacatacctacggacgtactccgtcgtgtatctaggtgttcccccttggcacgctttccacctgcgacaacgcggcctcagatcccgacctcgaacccccccccccccccccccaaacaacaacccagctcttcggctgtgcgcccgccaactcgacaaacaacaacatccaacaagtgcgaatttgaattcgactcgacagcccatcgattcgtctctcttcatgcgcatcaatccgatccggaaccgccgactttaacaacacccgtgccgggctcgaccacggggctcccgtagtccgccaaatacaggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 24 - DNA, 8196 bp, Artificial Sequence
  DNA sequence
ggcgcgccgtttaaacgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccccttttggaaagagcaccgcttctttttcgttcctcggcctgagccgtcgaatgcgaacgtcaaaaggcgaactagaaattctgaaacatagtacggattactccgtacccggttgttttgcaccgggattttgcttcaatcgccaccgagttccacccactttcgccaaggtacggattacagtaatccgtacatacctacggacgtactccgtcgtgtatctaggtgttcccccttggcacgctttccacctgcgacaacgcggcctcagatcccgacctcgaacccccccccccccccccccaaacaacaacccagctcttcggctgtgcgcccgccaactcgacaaacaacaacatccaacaagtgcgaatttgaattcgactcgacagcccatcgattcgtctctcttcatgcgcatcaatccgatccggaaccgccgactttaacaacacccgtgccgggctcgaccacggggctcccgtagtccgccaaatacatcgggtctgggatgtcttttttttattttatttttttatttttgtcgcggtgtgagtgtgtttgtcgggtccggttcggcagttcatgatcattcctctataaataaggtatggatcgtatatattatatattacatacagttgaagccttagcacagtatgaatctccatataaatctcttttttcttttcttttcttcttttttttttttttttgcaccccacccacgtgctttccttatattcatcatgcccttcatggctaggtgagttgataccaggactacgagatgtatatatatctcttgaacgattctcctagagtttgtttagacgtgcactgtcctctgataataataaatcagctgctgcattcatccacgtgcgaaaccagctttgttaggttcgaatgtagaccgttttggtatttcaaacggcagccattgcctccgcctttagaatctgtccaagctattgttcagcaactaatgtcaaaaaaaaaaaaaaaaaaacgcctaagcccccaacgtccggatagataagaatacagcagggtgacgggttggggggacggggaggttgtcttccgctgagcatgccaccacatcacatgaatgctttttcttcgctgcctggacctgaaccacccccggaggggctttcctccccccgcttgactactgcgctgacctccagacctcggacggatcctcaatggcggctaaccaggggtaagttcccatcaggctaccaccaccaccagaagggccggaactcgcgctccccgcgtccgaaacttcgccgtctctctcggtctcggcctcggtctcggtctcggcagaagcaccgtggccgcccccaatcaccatccacccgtccctcgtctcgcgaggatcggccgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 25 - DNA, 20 bp, Artificial Sequence
  primer
cctgcattgcaagttcccac

SEQ ID NO: 26 - DNA, 20 bp, Artificial Sequence
  primer
agtttgacagtgcccagagc

SEQ ID NO: 27 - DNA, 20 bp, Artificial Sequence
  primer
agcctggaaggcctatctgg

SEQ ID NO: 28 - DNA, 20 bp, Artificial Sequence
  primer
ggtcggattggcttggtaca

SEQ ID NO: 29 - DNA, 20 bp, Artificial Sequence
  primer
accaccgtcaacacgtacaa

SEQ ID NO: 30 - DNA, 20 bp, Artificial Sequence
  primer
caaaggtcttgccaccgatg

SEQ ID NO: 31 - DNA, 20 bp, Artificial Sequence
  primer
ttcgttgctaacactccccc

SEQ ID NO: 32 - DNA, 20 bp, Artificial Sequence
  primer
ctggttgatggccgagttga

SEQ ID NO: 33 - DNA, 20 bp, Artificial Sequence
  primer
ggcagattattccggaccgt

SEQ ID NO: 34 - DNA, 20 bp, Artificial Sequence
  primer
agtttgacagtgcccagagc

SEQ ID NO: 35 - DNA, 20 bp, Artificial Sequence
  primer
agcctggaaggcctatctgg

SEQ ID NO: 36 - DNA, 20 bp, Artificial Sequence
  primer
tcaacgtgtgggagcagtac

SEQ ID NO: 37 - DNA, 20 bp, Artificial Sequence
  primer
gggctccatctacgtcttcg

SEQ ID NO: 38 - DNA, 20 bp, Artificial Sequence
  primer
tggatccagggcgagtagaa

SEQ ID NO: 39 - DNA, 20 bp, Artificial Sequence
  primer
tgggctcgtacgacttcaac

SEQ ID NO: 40 - DNA, 20 bp, Artificial Sequence
  primer
cggcgatgttggagtcgtat

SEQ ID NO: 41 - DNA, 20 bp, Artificial Sequence
  primer
cgagaccgacaagaccaaca

SEQ ID NO: 42 - DNA, 20 bp, Artificial Sequence
  primer
gaagagcacgatgagcacga

SEQ ID NO: 43 - DNA, 20 bp, Artificial Sequence
  primer
ttggtaagacggtgcagatg

SEQ ID NO: 44 - DNA, 21 bp, Artificial Sequence
  primer
gtagttgatgcgttccttcca

SEQ ID NO: 45 - protein, 221 aa, Artificial sequence
  Amino acids
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSEPEA

SEQ ID NO: 46 - DNA, 666 bp, Artificial sequence
  DNA sequence
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggccctggcggcggcggcagcgagccggaggcctaa

SEQ ID NO: 47 - protein, 239 aa, Artificial sequence
  Amino acids
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSAHIVMVDAYKPTKGGGGSEPEA

SEQ ID NO: 48 - DNA, 720 bp, Artificial sequence
  DNA sequence
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtccaacctcaagccgttcgagcgcgacatctcgaccgagatctaccaggccggctccaccccctgcaacggcgtcgagggcttcaactgctacttccccctccagagctacggcttccagcccaccaacggcgtcggctaccagccctaccgcgtcgtcgtcctctccttcgagctcctgcacgccccggccaccgtctgcggccctggcggcggcggcagcgcccacatcgtcatggtcgacgcctacaagccgaccaagggcggcggcggctcggagcccgaggcctaa

SEQ ID NO: 49 - protein, 448 aa, Artificial sequence
  Amino acids
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - DNA, 1347 bp, Artificial sequence
  DNA sequence
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggccctggcggcggcggcagcggcggcggcggcagcgacaagacccacacctgcccgccctgccccgccccggaggccgctggcggccccagcgtcttcctcttcccgcccaagccgaaggacaccctgatgatctcgcgcaccccggaggtcacctgcgtcgtcgtcgacgtcagccacgaggacccggaggtcaagttcaactggtacgtcgacggcgtcgaggtccacaacgccaagaccaagccgcgcgaggagcagtacaactcgacctaccgcgtcgtctccgtcctcaccgtcctgcaccaggactggctcaacggcaaggagtacaagtgcaaggtctcgaacaaggccctgcccgccccgatcgagaagaccatctcgaaggccaagggccagccccgcgagccccaggtctacaccctcccgcccagccgcgacgagctgaccaagaaccaggtctcgctcacctgcctggtcaagggcttctacccctccgacatcgccgtcgagtgggagagcaacggccagccggagaacaactacaagaccaccccgcccgtcctggactccgacggctccttcttcctctacagcaagctgaccgtcgacaagtcgcgctggcagcagggcaacgtcttcagctgctcggtcatgcacgaggccctgcacaaccactacacccagaagtccctcagcctgtcgcccggctaa

SEQ ID NO: 51 - protein, 448 aa, Artificial sequence
  Amino acids
MYAKFATLAALVAGAAADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 52 - DNA, 1347 bp, Artificial sequence
  DNA sequence
atgtacgccaagttcgcgaccctcgccgcccttgtggctggcgccgctgctgacaagacccacacctgcccgccctgccccgccccggaggccgctggcggccccagcgtcttcctcttcccgcccaagccgaaggacaccctgatgatctcgcgcaccccggaggtcacctgcgtcgtcgtcgacgtcagccacgaggacccggaggtcaagttcaactggtacgtcgacggcgtcgaggtccacaacgccaagaccaagccgcgcgaggagcagtacaactcgacctaccgcgtcgtctccgtcctcaccgtcctgcaccaggactggctcaacggcaaggagtacaagtgcaaggtctcgaacaaggccctgcccgccccgatcgagaagaccatctcgaaggccaagggccagccccgcgagccccaggtctacaccctcccgcccagccgcgacgagctgaccaagaaccaggtctcgctcacctgcctggtcaagggcttctacccctccgacatcgccgtcgagtgggagagcaacggccagccggagaacaactacaagaccaccccgcccgtcctggactccgacggctccttcttcctctacagcaagctgaccgtcgacaagtcgcgctggcagcagggcaacgtcttcagctgctcggtcatgcacgaggccctgcacaaccactacacccagaagtccctcagcctgtcgcccggcggcggcggcggcagcggcggcggcggcagcaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccaccaacggcgtcggctaccagccctaccgcgtcgtcgtcctctccttcgagctcctgcacgcccccgccaccgtctgcggcccttaa

SEQ ID NO: 53 - protein, 219 aa, Artificial sequence
  Amino acids
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGSGEPEA

SEQ ID NO: 54 - DNA, 660 bp, Artificial sequence
  DNA sequence
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaagatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcgagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaa

SEQ ID NO: 55 - protein, 219 aa, Artificial sequence
  Amino acids
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSGEPEA

SEQ ID NO: 56 - DNA, 660 bp, Artificial sequence
  DNA sequence
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaacatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaa

SEQ ID NO: 57 - protein, 219 aa, Artificial sequence
  Amino acids
MYAKFATLAALVAGAAATNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGSGEPEA

SEQ ID NO: 58 - DNA, 660 bp, Artificial sequence
  DNA sequence
atgtacgccaagttcgcgaccctcgccgcccttgtggctggcgccgctgctaccaacctctgcccgttcggcgaggtcttcaacgccacccgcttcgcctccgtctacgcctggaaccgcaagcgcatctccaactgcgtcgccgactacagcgtcctgtacaacagcgcctcgttctccaccttcaagtgctacggcgtcagccccaccaagctcaacgacctgtgcttcaccaacgtctacgccgactccttcgtcatccgcggcgacgaggtccgccagatcgcccccggccagaccggcaccatcgccgactacaactacaagctccccgacgacttcaccggctgcgtcatcgcctggaacagcaacaacctggactcgaaggtcggcggcaactacaactacctctaccgcctgttccgcaagtcgaacctcaagccgttcgagcgcgacatctcgaccgagatctaccaggccggctccaccccctgcaacggcgtcaagggcttcaactgctacttccccctccagtcctacggcttccagcccacctacggcgtcggctaccagccctaccgcgtcgtcgtcctctccttcgagctcctgcacgcccccgccaccgtctgcggccctggcagcggcgagccggaggcctaa